(19) United States
(12) Patent Application Publication
(10) Pub. No.: US 2006/0167074 A1
(43) Pub. Date: Jul. 27, 2006

(54) Methods and compositions for the treatment of psychiatric disorders

(76) Inventor: Norbert Muller, Muenchen (DE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/320,757

(22) Filed: Dec. 30, 2005

Related U.S. Application Data
(63) Continuation-in-part of application No. 10/157,969, filed on May 31, 2002.
(60) Provisional application No. 60/364,904, filed on Mar. 14, 2002.

(30) Foreign Application Priority Data
Jun. 19, 2001 (DE) 101 29 328.8

(51) Int. Cl. A61K 31/415; A61K 31/365

(52) U.S. Cl. 514/406; 514/456; 514/471

(57) ABSTRACT

A method for the prevention, treatment, or inhibition of a psychiatric disorder, in particular schizophrenia, is described which comprises administering a COX-2 inhibitor or prodrug thereof to a subject. Moreover, a method for the prevention, treatment, or inhibition of a psychiatric disorder, in particular schizophrenia or depressive disorders, is disclosed comprising administering to a subject a COX-2 inhibitor or prodrug thereof in combination with a neuroleptic drug or an antidepressant. Compositions and kits that are suitable for the practice of the method are also described.

### CROSS-REFERENCE TO RELATED APPLICATIONS

This application is a continuation-in-part of U.S. patent application Ser. No. 10/157,969, filed May 31, 2002, which claims the benefit of U.S. Provisional Application No. 60/364,904, filed Mar. 14, 2002 and German Application No. 10129320.8, filed Jun. 19, 2001, which are incorporated herein by reference.

### FIELD OF THE INVENTION

The invention concerns a method for treating psychiatric disorders such as schizophrenia, delusional disorders, affective disorders, autism or tic disorders, in particular chronic schizophrenic psychoses and schizoaffective psychoses, temporary acute psychotic disorders, depressive episodes, recurring depressive episodes, manic episodes and bipolar affective disorders, which comprises administering a COX-2 (cyclooxygenase-2) inhibitor to a subject. Moreover, the invention provides a method and composition for treating psychiatric disorders such as schizophrenia, delusional disorders, affective disorders, autism or tic disorders which comprises administering a COX-2 inhibitor in combination with a neuroleptic drug or an antidepressant to a subject.

### BACKGROUND OF THE INVENTION

A relation between immunological dysfunctions and psychotic diseases, such as schizophrenia or affective disorders, has been discussed controversially over the last century.

In the case of schizophrenia for instance the pathogenesis is still unknown, but many findings indicate that schizophrenia is a syndrome based on different pathogenetic processes.

An inflammatory/immunological pathogenesis has been discussed for a subgroup of schizophrenic patients (Yolken R H, Torrey E F: Viruses, schizophrenia, and bipolar disorder. Clin Microbiol Rev 1995; 8:131-145; Körschenhausen D, Hampel H, Ackenheil M, Penning R, Müller N: Fibrin degradation products in post mortem brain tissue of schizophrenics: a possible marker for underlying inflammatory processes, Schizophr Res 1996; 19: 103-109; Müller N, Ackenheil M: Psychoneuroimmunology and the cytokine-network in the CNS: implications for psychiatric disorders. Prog Neuropsychopharmacol & Biol Psychiat 1998; 22: 1-33). Studies showed that activating cytokines like interleukin-1 (IL-1) and IL-2 are increased in the cerebrospinal fluid of schizophrenic patients compared to controls (Sirota P, Schild K, Elizur A, Djaldetti M, Fishman P: Increased Interleukin-1 and Interleukin-3 like activity in schizophrenic patients. Prog Neuropsychopharmacol & Biol Psychiatry 1995; 19: 85-83; Licinio J, Seibyl, J P, Altemus M, Chamey D S, Krystal J H: Elevated levels of Interleukin-2 in neuroleptic-free schizophrenics. AmJPsychiatry 1993; 150: 1408-1410), and that high levels of IL-2 in the cerebrospinal fluid are a predictor for the increased probability of a schizophrenic relapse (McAllister C G, van Kamen D P, Rehn T J, Miller A L, Gurklis J, Kelley M E, Yao J, Peters J L: Increases in CSF levels of Interleukin-2 in schizophrenia: effects of recurrence of psychosis and medication status. Am J Psychiatry 1995; 152: 1291-1297).

On the other hand, in a subgroup of schizophrenic patients a decreased immune response compared to controls has been observed, possibly due to a disturbance of antigen-presentation or antigen-recognition (Schwarz M J, Riedel M, Ackenheil M, Müller N: Decreased levels of soluble intercellular adhesion molecule −1 (sICAM-1) in unmedicated and medicated schizophrenic patients. Biol Psychiatry 2000; 47: 29-33), e.g. the increased immune reaction in the central nervous system may not be adequately regulated by an immune reaction in the peripheral immune system. This was observed mostly in acute schizophrenic patients presenting a recent onset of the disorder.

Another group of schizophrenic patients, however, seems to present an over-activation of the peripheral immune system in the sense of autoimmune processes (Radaport M H, Müller N: Immunological states associated with schizophrenia. In: Ader R, Felten D L, Cohen N (eds) Psychoneuroimmunology, Third Edition. Vol. 2, San Diego, Academic Press, 2001; pp 373-382; Radaport M H, McAllister C G, Kim Y S, Han J H, Pickar D, Nelson D M, Kirch D G, Paul S M: Increased soluble Interleukin-2 receptors in Caucasian and Korean schizophrenic patients. Biol Psychiatry 1994; 35: 767-771). In several studies, increased titers of antibodies against the heat-shock-protein 60 were observed (Kilidireas K, Latov N, Strauss D H, Aviva D G, Hashim G A, Gorman J M, Sadiq S A: Antibodies to human 60 KD hear-shock protein in patients with schizophrenia. Lancet 1992; 340: 569-572), the increase being accompanied by increased soluble IL-2 receptors in the serum and increased titers of the soluble adhesion molecule sICAM-1 (Radaport M H, Müller N: Immunological states associated with schizophrenia. In: Ader R, Felten D L, Cohen N (eds) Psychoneuroimmunology, Third Edition. Vol. 2, San Diego, Academic Press, 2001; pp 373-382; Schwarz M J, Riedel M, Gruber R, Ackenheil M, Müller N: Antibodies to heat-shock proteins in schizophrenic patients—Implications for disease mechanism. Am J Psychiatry 1999; 156, 1103, 1104). The close relationship between high sVCAM-1 titers and more pronounced schizophrenic negative symptoms (Schwarz M J, Riedel M, Gruber R, Ackenheil M, Müller N: Levels of soluble adhesion molecules in schizophrenia: Relation to psychopathology. In: N. Müller (Hrg) Psychiatry, Psychoneuroimmunology, and Viruses. Springer Verlag Wien, 1999; NY, pp. 121-130) as well as between high IgG levels in the cerebrospinal fluid and more pronounced negative symptoms further support this observation (Müller N, Ackenheil M: Immunoglobulin and albumin contents of cerebrospinal fluid in schizophrenic patients: The relationship to negative sympomatology. Schizophrenia Res 1995; 14: 223-228).

Affective diseases, in particular depressive diseases, may also have an inflammatory genesis. This is manifested in the fact that general inflammatory diseases are accompanied by depressive syndromes to an increased extent as well as in the fact that in depressive diseases, signs of inflammation occur more frequently in comparison to psychologically healthy persons. Scientifically, this was expressed in the monocyte/macrophage hypothesis of depression.

The occurrence of tics as well as of autism has also been discussed in many cases as a consequence of inflammatory processes.

The invention is based on the idea that substances with immunomodulatory properties could be used for the treatment of psychiatric disorders such as schizophrenia, delusional disorders, affective disorders, autism or tic disorders, which are at least partially based on immunological pathogenetic processes.

Recently, significant progress has been made in the field of inflammation and the development of drugs for the treatment of the inflammation-related disorders of osteoarthritis and rheumatoid arthritis. It has been known for some time that many of the common non-steroidal antiinflammatory drugs (NSAIDs) NSAIDs modulate prostaglandin synthesis by inhibition of cyclooxygenases that catalyze the transformation of arachidonic acid—the first step in the prostaglandin synthesis pathway. However, the use of high doses of many common NSAIDs can produce severe side effects that limit their therapeutic potential. In an effort to reduce the unwanted side effects of common NSAIDS, it was discovered that two cyclooxygenases are involved in the transformation of arachidonic acid as the first step in the prostaglandin synthesis pathway. These enzymes have been termed cyclooxygenase-1 (COX-1) and cyclooxygenase-2 (COX-2)(Needleman, P. et al., J. Rheumatol., 24, Suppl. 49:6-8 (1997); Fu, J. Y., et al., J. Biol. Chem., 265(28):16737-40 (1990)). COX-1 has been shown to be a constitutively produced enzyme that is involved in many of the non-inflammatory regulatory functions associated with prostaglandins. COX-2, on the other hand, is an inducible enzyme having significant involvement in the inflammatory process. Inflammation causes the induction of COX-2, leading to the release of prostanoids, which sensitize peripheral nociceptor terminals and produce localized pain hypersensitivity (Samad, T. A. et al., Nature, 410(6827):471-5 (2001)). Many of the common NSAIDs are now known to be inhibitors of both COX-1 and COX-2. Accordingly, when administered in sufficiently high levels, these NSAIDs affect not only the inflammatory consequences of COX-2 activity, but also the beneficial activities of COX-1. Recently, compounds that selectively inhibit COX-2 to a greater extent than the activity of COX-1 have been discovered. These new COX-2 inhibitors are believed to offer advantages that include the capacity to prevent or reduce inflammation while avoiding harmful side effects associated with the inhibition of COX-1, such as gastrointestinal and renal side effects, as well as inhibition of thrombocyte aggregation.

The use of COX-2 inhibitors in the therapy of arthritis and related indications is known. U.S. Pat. No. 5,760,068 describes the use of COX-2 inhibitors for the treatment of rheumatoid arthritis and osteoarthritis. WO 00/32189 discloses the preparation of pharmaceutical compositions containing the COX-2 inhibitor celecoxib and the use of celecoxib for the treatment of rheumatoid arthritis or as a painkiller.

Recently, progress has been made in the field of psychiatric disorders. For example, in the treatment of schizophrenia, a number of neuroleptic drugs (so-called classical and atypical neuroleptics) have become available, among which the more recent atypical neuroleptics excel by comparatively good effectiveness with a more favorable side effect profile. Unlike the classical neuroleptics, which are mainly effective for treating the positive symptoms of schizophrenia, the atypical neuroleptics improve both positive symptoms (hallucinations, delusions, and conceptual disorganization) and negative symptoms (apathy, social withdrawal, affective flattening, and poverty of speech) of schizophrenia. Plus, presumably due to their altered receptor binding profile, the atypicals cause minimal extrapyramidal symptoms and rarely cause tardive dyskinesias. Anyhow, neuroleptics in general act as syndrome oriented therapy and less as a causal therapy.

Therefore, a need exists for improved methods and compositions for the treatment of psychiatric disorders such as schizophrenia, delusional disorders, affective disorders, autism or tic disorders. In particular, it would be useful if such methods of treatment for disorders such as schizophrenia could be provided so that they reduced or avoided unwanted side effects.

### SUMMARY OF THE INVENTION

Therefore, the present invention is directed to a novel method for the prevention, treatment, or inhibition of a psychiatric disorder such as schizophrenia, delusional disorders, affective disorders, autism or tic disorders in a subject in need of such prevention, treatment, or inhibition, the method comprising administering to the subject a COX-2 inhibitor or prodrug thereof.

Furthermore, the invention is concerned with a novel method for the prevention, treatment, or inhibition of a psychiatric disorder in a subject in need of such prevention, treatment, or inhibition, the method comprising administering to the subject a neuroleptic agent or antidepressant and a COX-2 inhibitor or prodrug thereof.

The invention is also directed to a novel composition for the treatment, prevention, or inhibition of a psychiatric disorder comprising a neuroleptic agent or antidepressant and a COX-2 inhibitor or prodrug thereof.

The invention is further directed to a novel kit that is suitable for use in the treatment of psychiatric disorders such as schizophrenia, delusional disorders, affective disorders, autism or tic disorders, the kit comprising a first dosage form comprising a neuroleptic or antidepressant and a second dosage form comprising a COX-2 inhibitor, in quantities which comprise a therapeutically effective amount of the combination of the compounds for the treatment, prevention, or inhibition of a psychiatric disorder, for simultaneous, separate or sequential administration.

Among the several advantages found to be achieved by the present invention, therefore, may be noted the provision of treatment methods for psychiatric disorders that comprise administering COX-2 inhibitors, the provision of such methods and compositions that combine the effectiveness of neuroleptic agents or antidepressants and COX-2 inhibitors, and the provision of such methods and compositions for the treatment of disorders such as schizophrenia that can reduce or avoid unwanted side effects.

### BRIEF DESCRIPTION OF THE DRAWINGS

FIG. 1 shows the comparison of the PANSS score during treatment with risperidone-celecoxib or risperidone-placebo.

FIG. 2 shows the comparison of the PANSS negative score during treatment with risperidone-celecoxib or risperidone-placebo.

FIG. 3 shows the comparison of the PANSS global score during treatment with risperidone-celecoxib or risperidone-placebo.

FIG. 4 shows the plasma levels of risperidone and 9-OH-risperidone during treatment with risperidone-celecoxib or risperidone-placebo.

FIG. 5 shows the biperiden and benzodiazepine use during treatment with risperidone-celecoxib or risperidone-placebo.

FIG. 6 shows the effect of oral administration of rofecoxib on the behaviour of mice in the forced swim test. n=8 per group.

FIG. 7 shows the effect of oral administration of valdecoxib on the behaviour of mice in the forced swim test. n=9 for the control group, n=9 for the group treated with 4 mg/day valdecoxib, n=10 for the group treated with 20 mg/day valdecoxib.

FIG. 8 shows the effect of oral administration of etoricoxib on the behaviour of mice in the forced swim test. n=9 for the control group, n=10 for the group treated with 4 mg/day etoricoxib, n=9 for the group treated with 20 mg/day etoricoxib.

FIG. 9 shows the effect of oral administration of piroxicam on the behaviour of mice in the forced swim test. n=9 for the control group, n=10 for the group treated with 5 mg/day piroxicam.

FIG. 10 shows the comparison of HamD scores during therapy with celecoxib or placebo (ANOVA, estimated marginal means; advantage of celecoxib-group: Greenhouse-Geisser-corrected F=3.220; df 2.434; p 0.035).

### DETAILED DESCRIPTION OF THE INVENTION

In accordance with the present invention, it has been discovered that psychiatric disorders can be prevented, treated or inhibited in subjects in need of such prevention, treatment, or inhibition by administering to the subject a COX-2 inhibitor or prodrug thereof. Furthermore, it was discovered that psychiatric disorders can be prevented, treated or inhibited in subjects in need of such prevention, treatment, or inhibition by administering to the subject a neuroleptic agent or antidepressant in combination with a COX-2 inhibitor or prodrug thereof. It is believed that the novel combination of the neuroleptic agent or antidepressant and the COX-2 inhibitor are as efficacious as, and, in preferred embodiments, superior to, known and existing medications and treatment methods for psychiatric disorders, and that they offer such efficacy with reduced undesirable side effects. Kits that contain the novel combinations of a neuroleptic agent or antidepressant and the COX-2 inhibitor are also considered to be a part of the present invention.

The COX-2 inhibitors used in the present invention belong to the class of nonsteroidal anti-inflammatory drugs (NSAIDs). The term COX-2 inhibitor embraces compounds which selectively inhibit cyclooxygenase-2 over cyclooxygenase-1, and also includes pharmaceutically acceptable salts thereof. Also included within the scope of the present invention are compounds that act as prodrugs of cyclooxygenase-2-selective inhibitors. As used herein in reference to COX-2 inhibitors, the term “prodrug” refers to a chemical compound that can be converted into an active COX-2 inhibitor by metabolic or simple chemical processes within the body of the subject.

The COX-2 inhibitor of the present invention can be, for example, the COX-2 inhibitor meloxicam, Formula B-1 (CAS registry number 71125-38-7), or a pharmaceutically acceptable salt or prodrug thereof.

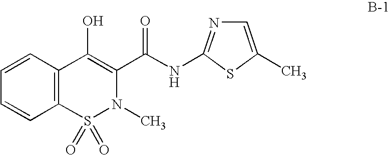

In another embodiment of the invention the COX-2 inhibitor can be the COX-2 inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

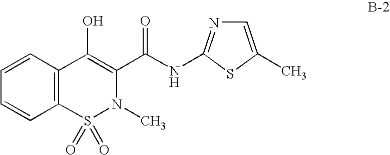

In a preferred embodiment of the invention the COX-2 inhibitor is a chromene derivative, that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, or III, shown below, and possessing, by way of example and not limitation, the structures disclosed in Table 1, including the diastereomers, enantiomers, racemates, tautomers, salts, esters, amides and prodrugs thereof.

Benzopyran COX-2 inhibitors useful in the practice of the present invention are described in U.S. Pat. Nos. 6,034,256 and 6,077,850.

Formula I is:

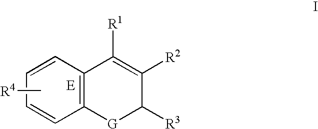

wherein G is selected from the group consisting of O or S or NRa;

wherein Ra is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein R⁴ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosufonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with ring E forms a naphthyl radical; or an isomer or pharmaceutically acceptable salt thereof; and

including the diastereomers, enantiomers, racemates, tautomers, salts, esters, amides and prodrugs thereof.

Formula II is:

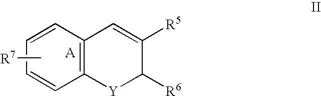

wherein:

Y is selected from the group consisting of O or S or NR^{b};

R^{b} is alkyl;

R⁵ is selected from the group consisting of carboxyl, aminocarbonyl, alkylsulfonylaminocarbonyl and alkoxycarbonyl;

R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;

or an isomer or pharmaceutically acceptable salt thereof.

The COX-2 inhibitor may also be a compound of Formula II, wherein:

Y is selected from the group consisting of oxygen and sulfur;

R⁵ is selected from the group consisting of carboxyl, lower alkyl, lower aralkyl and lower alkoxycarbonyl;

R⁶ is selected from the group consisting of lower haloalkyl, lower cycloalkyl and phenyl; and

R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R⁷ together with ring A forms a naphthyl radical;

or an isomer or pharmaceutically acceptable salt thereof.

The COX-2 inhibitor may also be a compound of Formula II, wherein:

R⁵ is carboxyl;

R⁶ is lower haloalkyl; and

R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;

or an isomer or pharmaceutically acceptable salt thereof.

The COX-2 inhibitor may also be a compound of Formula II, wherein:

R⁶ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;

or an isomer or pharmaceutically acceptable salt thereof.

The COX-2 inhibitor may also be a compound of Formula II, wherein:

R⁶ is selected from the group consisting trifluoromethyl and pentafluoroethyl; and

R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring A forms a naphthyl radical; or an isomer or prodrug thereof.

The COX-2 inhibitor of the present invention can also be a compound having the structure of Formula III:

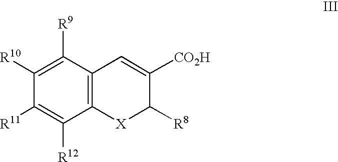

wherein:

X is selected from the group consisting of O and S;

R⁸ is lower haloalkyl;

R⁹ is selected from the group consisting of hydrido, and halo;

R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R¹¹ is selected from the group consisting of hydrido, lower alkyl, halo, lower alkoxy, and aryl; and

R¹² is selected from the group consisting of the group consisting of hydrido, halo, lower alkyl, lower alkoxy, and aryl;

or an isomer or prodrug thereof.

The COX-2 inhibitor can also be a compound of having the structure of Formula III, wherein

R⁸ is selected from the group consisting of trifluoromethyl and pentafluoroethyl;

R⁹ is selected from the group consisting of hydrido, chloro, and fluoro;

R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R¹¹ is selected from the group consisting of hydrido, methyl, ethyl, isopropyl, tert-butyl, chloro, methoxy, diethylamino, and phenyl; and

R¹² is selected from the group consisting of hydrido, chloro, bromo, fluoro, methyl, ethyl, tert-butyl, methoxy, and phenyl;

or an isomer or prodrug thereof.

TABLE 1
Examples of Chromene COX-2 Inhibitors as Embodiments

| Compound Number | Structural Formula |
|---|---|
| B-3 | 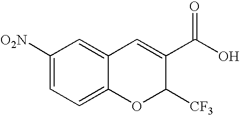 |
| | 6-Nitro-2-trifluoromethyl-2H-1- |
| | benzopyran-3-carboxylic acid |
| B-4 | 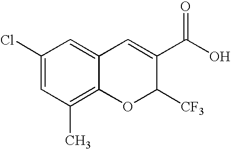 |
| | 6-Chloro-8-methyl-2-trifluoromethyl- |
| | 2H-1-benzopyran-3-carboxylic acid |
| B-5 | 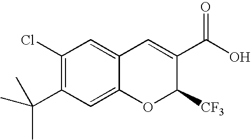 |
| | ((S)-6-Chloro-7-(1,1-dimethylethyl)-2-(trifluo |
| | romethyl-2H-1-benzopyran-3-carboxylic acid |
| B-6 | 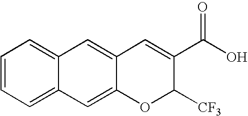 |
| | 2-Trifluoramethyl-2H-naphtho[2,3-b] |
| | pyran-3-carboxyiic acid |
| B-7 | 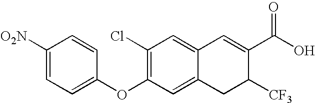 |
| | 6-Chloro-7-(4-nitrophenoxy)-2-(trifluoromethyl)- |
| | 2H-1-benzopyran-3-carboxylic acid |
| B-8 | 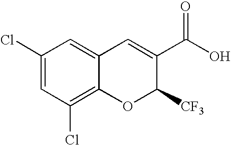 |
| | ((S)-6,8-Dichloro-2-(trifluoromethyl)- |
| | 2H-1-benzopyran-3-carboxylic acid |
| B-9 | 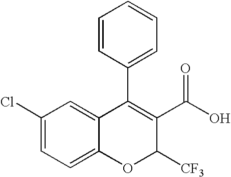 |
| | 6-Chloro-2-(trifluoromethyl)-4-phenyl-2H- |
| | 1-benzopyran-3-carboxylic acid |
| B-10 | 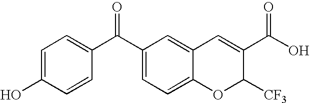 |
| | 6-(4-Hydroxybenzoyl)-2-(trifluoromethyl)- |
| | 2H-1-benzopyran-3-carboxylic acid |
| B-11 | 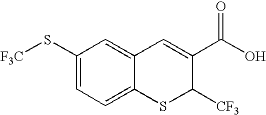 |
| | 2-(Trifluoromethyl)-6-[(trifluoromethyl)thio] |
| | 2H-1-benzothiopyran-3-carboxylic acid |
| B-12 | 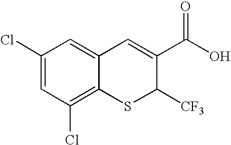 |
| | 6,8-Dichloro-2-trifluoromethyl-2H-1- |
| | benzothiopyran-3-carboxylic acid |
| B-13 | 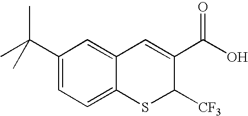 |
| | 6-(1,1-Dimethylethyl)-2-(trifluoromethyl)- |
| | 2H-1-benzothiopyran-3-carboxylic acid |
| B-14 | 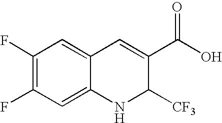 |
| | 6,7-Difluoro-1,2-dihydro-2-(trifluoro |
| | methyl)-3-quinolinecarboxylic acid |
| B-15 | 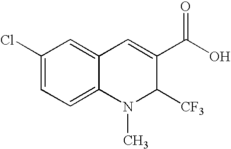 |
| | 6-Chloro-1,2-dihydro-1-methyl-2-(trifluoro |
| | methyl)-3-quinolinecarboxylic acid |
| B-16 | 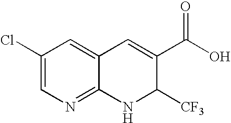 |
| | 6-Chloro-2-(trifluoromethyl)-1,2-dihydro |
| | [1,8]naphthyridine-3-carboxylic acid |
| B-17 | 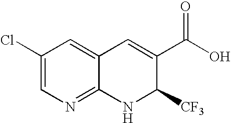 |
| | ((S)-6-Chloro-1,2-dihydro-2-(trifluoro |
| | methyl)-3-quinolinecarboxylic acid |

Specific compounds that are useful for the COX-2 inhibitor include:
a1) 8-acetyl-3-(4-fluorophenyl)-2-(4-methylsulfonyl)phenyl-imidazo(1,2-a)pyridine;
a2) 5,5-dimethyl-4-(4-methylsulfonyl)phenyl-3-phenyl-2-(5H)-furanone;
a3) 5-(4-fluorophenyl)-1-[4-(methylsulfonyl)phenyl]-3-(trifluoromethyl)pyrazole;
a4) 4-(4-fluorophenyl)-5-[4-(methylsulfonyl)phenyl]-1-phenyl-3-(trifluoromethyl)pyrazole;
a5) 4-(5-(4-chlorophenyl)-3-(4-methoxyphenyl)-1H-pyrazol-1-yl)benzenesulfonamide;
a6) 4-(3,5-bis(4-methylphenyl)-1H-pyrazol-1-yl)benzenesulfonamide;
a7) 4-(5-(4-chlorophenyl)-3-phenyl-1H-pyrazol-1-yl)benzenesulfonamide;
a8) 4-(3,5-bis(4-methoxyphenyl)-1H-pyrazol-1-yl)benzenesulfonamide;
a9) 4-(5-(4-chlorophenyl)-3-(4-methylphenyl)-1H-pyrazol-1-yl)benzenesulfonamide;
a10) 4-(5-(4-chlorophenyl)-3-(4-nitrophenyl)-1H-pyrazol-1-yl)benzenesulfonamide;
b1) 4-(5-(4-chlorophenyl)-3-(5-chloro-2-thienyl)-1H-pyrazol-1-yl)benzenesulfonamide;
b2) 4-(4-chloro-3,5-diphenyl-1H-pyrazol-1-yl)benzenesulfonamide
b3) 4-[5-(4-chlorophenyl)-3-(trifluoromethyl)-1H-pyrazol-1-yl]benzenesulfonamide;
b4) 4-[5-phenyl-3-(trifluoromethyl)-1H-pyrazol-1-yl]benzenesulfonamide;
b5) 4-[5-(4-fluorophenyl)-3-(trifluoromethyl)-1H-pyrazol-1-yl]benzenesulfonamide;
b6) 4-[5-(4-methoxyphenyl)-3-(trifluoromethyl)-1H-pyrazol-1-yl]benzenesulfonamide;
b7) 4-[5-(4-chlorophenyl)-3-(difluoromethyl)-1H-pyrazol-1-yl]benzenesulfonamide;
b8) 4-[5-(4-methylphenyl)-3-(trifluoromethyl)-1H-pyrazol-1-yl]benzenesulfonamide;
b9) 4-[4-chloro-5-(4-chlorophenyl)-3-(trifluoromethyl)-1H-pyrazol-1-yl]benzenesulfonamide;
b10) 4-[3-(difluoromethyl)-5-(4-methylphenyl)-1H-pyrazol-1-yl]benzenesulfonamide;
c1) 4-[3-(difluoromethyl)-5-phenyl-1H-pyrazol-1-yl]benzenesulfonamide;
c2) 4-[3-(difluoromethyl)-5-(4-methoxyphenyl)-1H-pyrazol-1-yl]benzenesulfonamide;
c3) 4-[3-cyano-5-(4-fluorophenyl)-1H-pyrazol-1-yl]benzenesulfonamide;
c4) 4-[3-(difluoromethyl)-5-(3-fluoro-4-methoxyphenyl)-1H-pyrazol-1-yl]benzenesulfonamide;
c5) 4-[5-(3-fluoro-4-methoxyphenyl)-3-(trifluoromethyl)-1H-pyrazol-1-yl]benzenesulfonamide;
c6) 4-[4-chloro-5-phenyl-1H-pyrazol-1-yl]benzenesulfonamide;
c7) 4-[5-(4-chlorophenyl)-3-(hydroxymethyl)-1H-pyrazol-1-yl]benzenesulfonamide;
c8) 4-[5-(4-(N,N-dimethylamino)phenyl)-3-(trifluoromethyl)-1H-pyrazol-1-yl]benzenesulfonamide;
c9) 5-(4-fluorophenyl)-6-[4-(methylsulfonyl)phenyl]spiro[2.4]hept-5-ene;
c10) 4-[6-(4-fluorophenyl)spiro[2.4]hept-5-en-5-yl]benzenesulfonamide;
d1) 6-(4-fluorophenyl)-7-[4-(methylsulfonyl)phenyl]spiro[3.4]oct-6-ene;
d2) 5-(3-chloro-4-methoxyphenyl)-6-[4-(methylsulfonyl)phenyl]spiro[2.4]hept-5-ene;
d3) 4-[6-(3-chloro-4-methoxyphenyl)spiro[2.4]hept-5-en-5-yl]benzenesulfonamide;
d4) 5-(3,5-dichloro-4-methoxyphenyl)-6-[4-(methylsulfonyl)phenyl]spiro[2.4]hept-5-ene;
d5) 5-(3-chloro-4-fluorophenyl)-6-[4-(methylsulfonyl)phenyl]spiro[2.4]hept-5-ene;
d6) 4-[6-(3,4-dichlorophenyl)spiro[2.4]hept-5-en-5-yl]benzenesulfonamide;
d7) 2-(3-chloro-4-fluorophenyl)-4-(4-fluorophenyl)-5-(4-methylsulfonylphenyl)thiazole;
d8) 2-(2-chlorophenyl)-4-(4-fluorophenyl)-5-(4-methylsulfonylphenyl)thiazole;
d9) 5-(4-fluorophenyl)-4-(4-methylsulfonylphenyl)-2-methylthiazole;
d10) 4-(4-fluorophenyl)-5-(4-methylsulfonylphenyl)-2-trifluoromethylthiazole;
e1) 4-(4-fluorophenyl)-5-(4-methylsulfonylphenyl)-2-(2-thienyl)thiazole;
e2) 4-(4-fluorophenyl)-5-(4-methylsulfonylphenyl)-2-benzylaminothiazole;
e3) 4-(4-fluorophenyl)-5-(4-methylsulfonylphenyl)-2-(1-propylamino)thiazole;
e4) 2-[(3,5-dichlorophenoxy)methyl)-4-(4-fluorophenyl)-5-[4-(methylsulfonyl)phenyl]thiazole;
e5) 5-(4-fluorophenyl)-4-(4-methylsulfonylphenyl)-2-trifluoromethylthiazole;
e6) 1-methylsulfonyl-4-[1,1-dimethyl-4-(4-fluorophenyl)cyclopenta-2,4-dien-3-yl]benzene;
e7) 4-[4-(4-fluorophenyl)-1,1-dimethylcyclopenta-2,4-dien-3-yl]benzenesulfonamide;
e8) 5-(4-fluorophenyl)-6-[4-(methylsulfonyl)phenyl]spiro[2.4]hepta-4,6-diene;
e9) 4-[6-(4-fluorophenyl)spiro[2.4]hepta-4,6-dien-5-yl]benzenesulfonamide;
e10) 6-(4-fluorophenyl)-2-methoxy-5-[4-(methylsulfonyl)phenyl]-pyridine-3-carbonitrile;
f1) 2-bromo-6-(4-fluorophenyl)-5-[4-(methylsulfonyl)phenyl]-pyridine-3-carbonitrile;
f2) 6-(4-fluorophenyl)-5-[4-(methylsulfonyl)phenyl]-2-phenyl-pyridine-3-carbonitrile;
f3) 4-[2-(4-methylpyridin-2-yl)-4-(trifluoromethyl)-1H-imidazol-1-yl]benzenesulfonamide;
f4) 4-[2-(5-methylpyridin-3-yl)-4-(trifluoromethyl)-1H-imidazol-1-yl]benzenesulfonamide;
f5) 4-[2-(2-methylpyridin-3-yl)-4-(trifluoromethyl)-1H-imidazol-1-yl]benzenesulfonamide;
f6) 3-[1-[4-(methylsulfonyl)phenyl]-4-(trifluoromethyl)-1H-imidazol-2-yl]pyridine;
f7) 2-[1-[4-(methylsulfonyl)phenyl-4-(trifluoromethyl)-1H-imidazol-2-yl]pyridine;
f8) 2-methyl-4-[1-[4-(methylsulfonyl)phenyl-4-(trifluoromethyl)-1H-imidazol-2-yl]pyridine;
f9) 2-methyl-6-[1-[4-(methylsulfonyl)phenyl-4-(trifluoromethyl)-1H-imidazol-2-yl]pyridine;
f10) 4-[2-(6-methylpyridin-3-yl)-4-(trifluoromethyl)-1H-imidazol-1-yl]benzenesulfonamide;
g1) 2-(3,4-difluorophenyl)-1-[4-(methylsulfonyl)phenyl]-4-(trifluoromethyl)-1H-imidazole;
g2) 4-[2-(4-methylphenyl)-4-(trifluoromethyl)-1H-imidazol-1-yl]benzenesulfonamide;
g3) 2-(4-chlorophenyl)-1-[4-(methylsulfonyl)phenyl]-4-methyl-1H-imidazole;
g4) 2-(4-chlorophenyl)-1-[4-(methylsulfonyl)phenyl]-4-phenyl-1H-imidazole;
g5) 2-(4-chlorophenyl)-4-(4-fluorophenyl)-1-[4-(methylsulfonyl)phenyl]-1H-imidazole;
g6) 2-(3-fluoro-4-methoxyphenyl)-1-[4-(methylsulfonyl)phenyl-4-(trifluoromethyl)-1H-imidazole;
g7) 1-[4-(methylsulfonyl)phenyl]-2-phenyl-4-trifluoromethyl-1H-imidazole;
g8) 2-(4-methylphenyl)-1-[4-(methylsulfonyl)phenyl]-4-trifluoromethyl-1H-imidazole;
g9) 4-[2-(3-chloro-4-methylphenyl)-4-(trifluoromethyl)-1H-imidazol-1-yl]benzenesulfonamide;
g10) 2-(3-fluoro-5-methylphenyl)-1-[4-(methylsulfonyl)phenyl]-4-(trifluoromethyl)-1H-imidazole;
h1) 4-[2-(3-fluoro-5-methylphenyl)-4-(trifluoromethyl)-1H-imidazol-1-yl]benzenesulfonamide;
h2) 2-(3-methylphenyl)-1-[4-(methylsulfonyl)phenyl]-4-trifluoromethyl-1H-imidazole;
h3) 4-[2-(3-methylphenyl)-4-trifluoromethyl-1H-imidazol-1-yl]benzenesulfonamide;
h4) 1-[4-(methylsulfonyl)phenyl]-2-(3-chlorophenyl)-4-trifluoromethyl-1H-imidazole;
h5) 4-[2-(3-chlorophenyl)-4-trifluoromethyl-1H-imidazol-1-yl]benzenesulfonamide;
h6) 4-[2-phenyl-4-trifluoromethyl-1H-imidazol-1-yl]benzenesulfonamide;
h7) 4-[2-(4-methoxy-3-chlorophenyl)-4-trifluoromethyl-1H-imidazol-1-yl]benzenesulfonamide;
h8) 1-allyl-4-(4-fluorophenyl)-3-[4-(methylsulfonyl)phenyl]-5-(trifluoromethyl)-1H-pyrazole;
h10) 4-[1-ethyl-4-(4-fluorophenyl)-5-(trifluoromethyl)-1H-pyrazol-3-yl]benzenesulfonamide;
i1) N-phenyl-[4-(4-luorophenyl)-3-[4-(methylsulfonyl)phenyl]-5-(trifluoromethyl)-1H-pyrazol-1-yl]acetamide;
i2) ethyl[4-(4-fluorophenyl)-3-[4-(methylsulfonyl)phenyl]-5-(trifluoromethyl)-1H-pyrazol-1-yl]acetate;
i3) 4-(4-fluorophenyl)-3-[4-(methylsulfonyl)phenyl]-1-(2-phenylethyl)-1H-pyrazole;
i4) 4-(4-fluorophenyl)-3-[4-(methylsulfonyl)phenyl]-1-(2-phenylethyl)-5-(trifluoromethyl)pyrazole;
i5) 1-ethyl-4-(4-fluorophenyl)-3-[4-(methylsulfonyl)phenyl]-5-(trifluoromethyl)-1H-pyrazole;
i6) 5-(4-fluorophenyl)-4-(4-methylsulfonylphenyl)-2-trifluoromethyl-1H-imidazole;
i7) 4-[4-(methylsulfonyl)phenyl]-5-(2-thiophenyl)-2-(trifluoromethyl)-1H-imidazole;
i8) 5-(4-fluorophenyl)-2-methoxy-4-[4-(methylsulfonyl)phenyl]-6-(trifluoromethyl)pyridine;
i9) 2-ethoxy-5-(4-fluorophenyl)-4-[4-(methylsulfonyl)phenyl]-6-(trifluoromethyl)pyridine;
i10) 5-(4-fluorophenyl)-4-[4-(methylsulfonyl)phenyl]-2-(2-propynyloxy)-6-(trifluoromethyl)pyridine;
j1) 2-bromo-5-(4-fluorophenyl)-4-[4-(methylsulfonyl)phenyl]-6-(trifluoromethyl)pyridine;
j2) 4-[2-(3-chloro-4-methoxyphenyl)-4,5-difluorophenyl]benzenesulfonamide;
j3) 1-(4-fluorophenyl)-2-[4-(methylsulfonyl)phenyl]benzene;
j4) 5-difluoromethyl-4-(4-methylsulfonylphenyl)-3-phenylisoxazole;
j5) 4-[3-ethyl-5-phenylisoxazol-4-yl]benzenesulfonamide;
j6) 4-[5-difluoromethyl-3-phenylisoxazol-4-yl]benzenesulfonamide;
j7) 4-[5-hydroxymethyl-3-phenylisoxazol-4-yl]benzenesulfonamide;
j8) 4-[5-methyl-3-phenyl-isoxazol-4-yl]benzenesulfonamide;
j9) 1-[2-(4-fluorophenyl)cyclopenten-1-yl]-4-(methylsulfonyl)benzene;
j10) 1-[2-(4-fluoro-2-methylphenyl)cyclopenten-1-yl]-4-(methylsulfonyl)benzene;
k1) 1-[2-(4-chlorophenyl)cyclopenten-1-yl]-4-(methylsulfonyl)benzene;
k2) 1-[2-(2,4-dichlorophenyl)cyclopenten-1-yl]-4-(methylsulfonyl)benzene;
k3) 1-[2-(4-trifluoromethylphenyl)cyclopenten-1-yl]-4-(methylsulfonyl)benzene;
k4) 1-[2-(4-methylthiophenyl)cyclopenten-1-yl]-4-(methylsulfonyl)benzene;
k5) 1-[2-(4-fluorophenyl)-4,4-dimethylcyclopenten-1-yl]-4-(methylsulfonyl)benzene;
k6) 4-[2-(4-fluorophenyl)-4,4-dimethylcyclopenten-1-yl]benzenesulfonamide;
k7) 1-[2-(4-chlorophenyl)-4,4-dimethylcyclopenten-1-yl]-4-(methylsulfonyl)benzene;
k8) 4-[2-(4-chlorophenyl)-4,4-dimethylcyclopenten-1-yl]benzenesulfonamide;
k9) 4-[2-(4-fluorophenyl)cyclopenten-1-yl]benzenesulfonamide;
k10) 4-[2-(4-chlorophenyl)cyclopenten-1-yl]benzenesulfonamide;
l1) 1-[2-(4-methoxyphenyl)cyclopenten-1-yl]-4-(methylsulfonyl)benzene;
l2) 1-[2-(2,3-difluorophenyl)cyclopenten-1-yl]-4-(methylsulfonyl)benzene;
l3) 4-[2-(3-fluoro-4-methoxyphenyl)cyclopenten-1-yl]benzenesulfonamide;
l4) 1-[2-(3-chloro-4-methoxyphenyl)cyclopenten-1-yl]-4-(methylsulfonyl)benzene;
l5) 4-[2-(3-chloro-4-fluorophenyl)cyclopenten-1-yl]benzenesulfonamide;
l6) 4-[2-(2-methylpyridin-5-yl)cyclopenten-1-yl]benzenesulfonamide;
l7) ethyl 2-[4-(4-fluorophenyl)-5-[4-(methylsulfonyl)phenyl]oxazol-2-yl]-2-benzyl-acetate;
l8) 2-[4-(4-fluorophenyl)-5-[4-(methylsulfonyl)phenyl]oxazol-2-yl]acetic acid;
l9) 2-(tert-butyl)-4-(4-fluorophenyl)-5-[4-(methylsulfonyl)phenyl]oxazole;
l10) 4-(4-fluorophenyl)-5-[4-(methylsulfonyl)phenyl]-2-phenyloxazole;
m1) 4-(4-fluorophenyl)-2-methyl-5-[4-(methylsulfonyl)phenyl]oxazole; and
m2) 4-[5-(3-fluoro-4-methoxyphenyl)-2-trifluoromethyl-4-oxazolyl]benzenesulfonamide.
m3) 6-chloro-2-trifluoromethyl-2H-1-benzopyran-3-carboxylic acid;
m4) 6-chloro-7-methyl-2-trifluoromethyl-2H-1-benzopyran-3-carboxylic acid;
m5) 8-(1-methylethyl)-2-trifluoromethyl-21H-1-benzopyran-3-carboxylic acid;
m6) 6-chloro-7-(1,1-dimethylethyl)-2-trifluoromethyl-2H-1-benzopyran-3-carboxylic acid;
m7) 6-chloro-8-(1-methylethyl)-2-trifluoromethyl-2H-1-benzopyran-3-carboxylic acid;
m8) 2-trifluoromethyl-3H-naphthopyran-3-carboxylic acid;
m9) 7-(1,1-dimethylethyl)-2-trifluoromethyl-2H-1-benzopyran-3-carboxylic acid;
m10) 6-bromo-2-trifluoromethyl-2H-1-benzopyran-3-carboxylic acid;
n1) 8-chloro-2-trifluoromethyl-2H-1-benzopyran-3-carboxylic acid;
n2) 6-trifluoromethoxy-2-trifluoromethyl-2H-1-benzopyran-3-carboxylic acid;
n3) 5,7-dichloro-2-trifluoromethyl-2H-1-benzopyran-3-carboxylic acid;
n4) 8-phenyl-2-trifluoromethyl-2H-1-benzopyran-3-carboxylic acid;
n5) 7,8-dimethyl-2-trifluoromethyl-2H-1-benzopyran-3-carboxylic acid;
n6) 6,8-bis(dimethylethyl)-2-trifluoromethyl-2H-1-benzopyran-3-carboxylic acid;
n7) 7-(1-methylethyl)-2-trifluoromethyl-2H-1-benzopyran-3-carboxylic acid;
n8) 7-phenyl-2-trifluoromethyl-2H-1-benzopyran-3-carboxylic acid;
n9) 6-chloro-7-ethyl-2-trifluoromethyl-2H-1-benzopyran-3-carboxylic acid;
n10) 6-chloro-8-ethyl-2-trifluoromethyl-2H-1-benzopyran-3-carboxylic acid;
o1) 6-chloro-7-phenyl-2-trifluoromethyl-2H-1-benzopyran-3-carboxylic acid;
o2) 6,7-dichloro-2-trifluoromethyl-2H-1-benzopyran-3-carboxylic acid;
o3) 6,8-dichloro-2-trifluoromethyl-2H-1-benzopyran-3-carboxylic acid;
o4) 2-trifluoromethyl-3H-naptho[2,1-b]pyran-3-carboxylic acid;
o5) 6-chloro-8-methyl-2-trifluoromethyl-2H-1-benzopyran-3-carboxylic acid;
o6) 8-chloro-6-methyl-2-trifluoromethyl-2H-1-benzopyran-3-carboxylic acid;
o7) 8-chloro-6-methoxy-2-trifluoromethyl-2H-1-benzopyran-3-carboxylic acid;
o8) 6-bromo-8-chloro-2-trifluoromethyl-2H-1-benzopyran-3-carboxylic acid;
o9) 8-bromo-6-fluoro-2-trifluoromethyl-2H-1-benzopyran-3-carboxylic acid;
o10) 8-bromo-6-methyl-2-trifluoromethyl-2H-1-benzopyran-3-carboxylic acid;
p1) 8-bromo-5-fluoro-2-trifluoromethyl-2H-1-benzopyran-3-carboxylic acid;
p2) 6-chloro-8-fluoro-2-trifluoromethyl-2H-1-benzopyran-3-carboxylic acid;
p3) 6-bromo-8-methoxy-2-trifluoromethyl-2H-1-benzopyran-3-carboxylic acid;
p4) 6-[[(phenylmethyl)amino]sulfonyl]-2-trifluoromethyl-2H-1-benzopyran-3-carboxylic acid;
p5) 6-[(dimethylamino)sulfonyl]-2-trifluoromethyl-2H-1-benzopyran-3-carboxylic acid;
p6) 6-[(methylamino)sulfonyl]-2-trifluoromethyl-2H-1-benzopyran-3-carboxylic acid;
p7) 6-[(4-morpholino)sulfonyl]-2-trifluoromethyl-2H-1-benzopyran-3-carboxylic acid;
p8) 6-[(1,1-dimethylethyl)aminosulfonyl]-2-trifluoromethyl-2H-1-benzopyran-3-carboxylic acid;
p9) 6-[(2-methylpropyl)aminosulfonyl]-2-trifluoromethyl-2H-1-benzopyran-3-carboxylic acid;
p10) 6-methylsulfonyl-2-trifluoromethyl-2H-1-benzopyran-3-carboxylic acid;
q1) 8-chloro-6-[[(phenylmethyl)amino]sulfonyl]-2-trifluoromethyl-2H-1-benzopyran-3-carboxylic acid;
q2) 6-phenylacetyl-2-trifluoromethyl-2H-1-benzopyran-3-carboxylic acid;
q3) 6,8-dibromo-2-trifluoromethyl-2H-1-benzopyran-3-carboxylic acid;
q4) 8-chloro-5,6-dimethyl-2-trifluoromethyl-2H-1-benzopyran-3-carboxylic acid;
q5) 6,8-dichloro-(S)-2-trifluoromethyl-2H-1-benzopyran-3-carboxylic acid;
q6) 6-benzylsulfonyl-2-trifluoromethyl-2H-1-benzopyran-3-carboxylic acid;
q7) 6-[[N-(2-furylmethyl)amino]sulfonyl]-2-trifluoromethyl-2H-1-benzopyran-3-carboxylic acid;
q8) 6-[[N-(2-phenylethyl)amino]sulfonyl]-2-trifluoromethyl-2H-1-benzopyran-3-carboxylic acid;
q9) 6-iodo-2-trifluoromethyl-2H-1-benzopyran-3-carboxylic acid;
q10) 7-(1,1-dimethylethyl)-2-pentafluoroethyl-2H-1-benzopyran-3-carboxylic acid;
r1) 5,5-dimethyl-3-(3-fluorophenyl)-4-(4-methyl-sulphonyl-2(5H)-fluranone;
r2) 6-chloro-2-trifluoromethyl-2H-1-benzothiopyran-3-carboxylic acid;
r3) 4-[5-(4-chlorophenyl)-3-(trifluoromethyl)-1H-pyrazol-1-yl]benzene sulfonamide;
r4) 4-[5-(4-methylphenyl)-3-(trifluoromethyl)-1H-pyrazol-1-yl]benzenesulfonamide;
r5) 4-[5-(3-fluoro-4-methoxyphenyl)-3-(difluoromethyl)-1H-pyrazol-1-yl]benzenesulfonamide;
r6) 3-[1-[4-(methylsulfonyl)phenyl]-4-trifluoromethyl-1H-imidazol-2-yl]pyridine;
r7) 2-methyl-5-[1-[4-(methylsulfonyl)phenyl]-4-trifluoromethyl-1H-imidazol-2-yl]pyridine;
r8) 4-[2-(5-methylpyridin-3-yl)-4-(trifluoromethyl)-1H-imidazol-1-yl]benzenesulfonamide;
r9) 4-[5-methyl-3-phenylisoxazol-4-yl]benzenesulfonamide;
r10) 4-[5-hydroxymethyl-3-phenylisoxazol-4-yl]benzenesulfonamide;
s1) [2-trifluoromethyl-5-(3,4-difluorophenyl)-4-oxazolyl]benzenesulfonamide;
s2) 4-[2-methyl-4-phenyl-5-oxazolyl]benzenesulfonamide; or
s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic COX-2 inhibitors represented by the general structure of Formula IV:

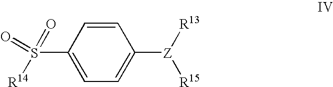

wherein:

Z is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the COX-2 inhibitor represented by the above Formula IV is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the COX-2 inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Inhibitors as Embodiments

| | |
|---|---|
| Compound Number | Structural Formula |
| B-18 | 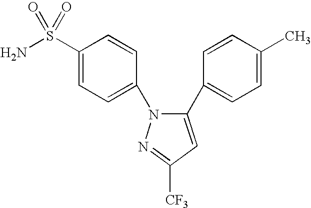 |
| B-19 | 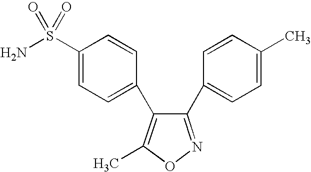 |
| B-20 | 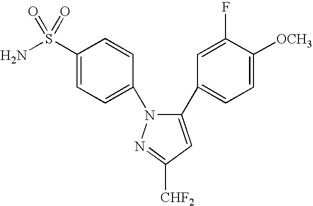 |
| B-21 | 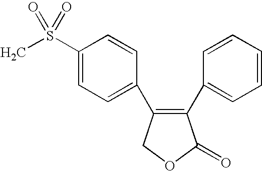 |
| B-22 | 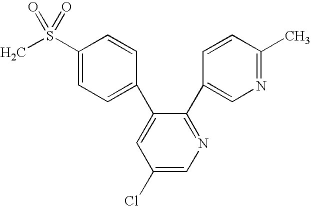 |
| B-23 | 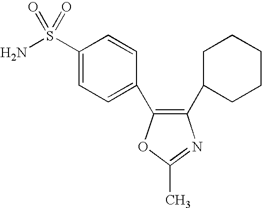 |

In a more preferred embodiment of the invention, the COX-2 inhibitor is selected from the group consisting of celecoxib, rofecoxib and etoricoxib. In a preferred embodiment of the invention, parecoxib (U.S. Pat. No. 5,932,598), having the structure shown in B-24, which is a therapeutically effective prodrug of the tricyclic COX-2 inhibitor valdecoxib, B-19, (U.S. Pat. No. 5,633,272), may be advantageously employed as a source of a cyclooxygenase inhibitor. A preferred form of parecoxib is sodium parecoxib.

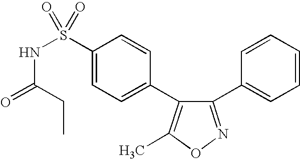

In another preferred embodiment of the invention, the compound ABT-963 having the formula B-25 that has been previously described in International Publication number WO 00/24719, is another tricyclic COX-2 inhibitor which may be advantageously employed.

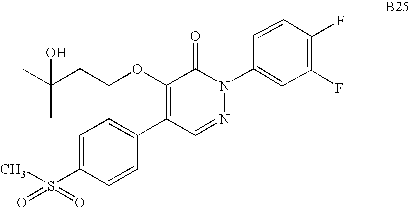

In a further preferred embodiment of the invention the cyclooxygenase inhibitor can be selected from the class of phenylacetic acid derivative COX-2 inhibitors represented by the general structure of Formula V:

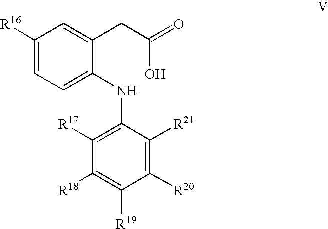

wherein R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

A particularly preferred phenylacetic acid derivative COX-2 inhibitor that is described in WO 99/11605 is a compound that has the designation of COX189 (CAS RN 346670-74-4), and that has the structure shown in Formula V,

wherein R¹⁶ is ethyl;

R¹⁷ and R¹⁹ are chloro;

R¹⁸ and R²⁰ are hydrogen; and

R²¹ is methyl.

Compounds that have a structure similar to that shown in Formula V, which can serve as the COX-2 inhibitor of the present invention, are described in U.S. Pat. Nos. 6,310,099 and 6,291,523.

Other preferred COX-2 inhibitors that can be used in the present invention have the general structure shown in formula VI, where the J group is a carbocycle or a heterocycle. Particularly preferred embodiments have the structure:

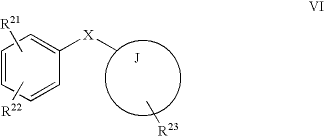

wherein:

X is O; J is 1-phenyl; R²¹ is 2-NHSO₂CH₃; R²² is 4-NO₂; and there is no R²³ group, (nimesulide); and

X is O; J is 1-oxo-inden-5-yl; R²¹ is 2-F; R²² is 4-F; and R²³ is 6-NHSO2CH3, (flosulide); and

X is O; J is cyclohexyl; R²¹ is 2-NHSO₂CH₃; R²² is 5-NO₂; and there is no R²³ group, (NS-398); and

X is S; J is 1-oxo-inden-5-yl; R²¹ is 2-F; R²² is 4-F; and R²³ is 6-N—SO₂CH₃ Na⁺, (L-745337); and

X is S; J is thiophen-2-yl; R²¹ is 4-F; there is no R²² group; and R²³ is 5-NHSO₂CH₃, (RWJ-63556); and

X is O; J is 2-oxo-5(R)-methyl-5-(2,2,2-trifluoroethyl)furan-(5H)-3-yl; R²¹ is 3-F; R²² is 4-F; and R²³ is 4-(p-SO₂CH₃)C₆H₄, (L-784512).

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J. P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

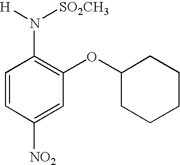

An evaluation of the antiinflammatory activity of the COX-2 inhibitor, RWJ 63556, in a canine model of inflammation, was described by Kirchner et al., in J Pharmacol Exp Ther 282, 1094-1101 (1997).

Other materials that can serve as he COX-2 inhibitor of the present invention include diarylmethylidenefuran derivatives that are described in U.S. Pat. No. 6,180,651. Such diarylmethylidenefuran derivatives have the general formula shown below in formula VII:

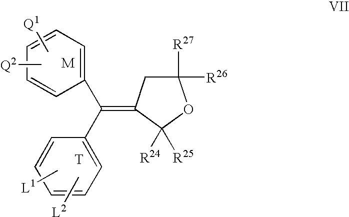

wherein:

the rings T and M independently are:

a phenyl radical,

a naphthyl radical,

a radical derived from a heterocycle comprising 5 to 6 members and possessing from 1 to 4 heteroatoms, or a radical derived from a saturated hydrocarbon ring having from 3 to 7 carbon atoms;

at least one of the substituents Q¹, Q², L¹ or L² is:

an —S(O)ₙ—R group, in which n is an integer equal to 0, 1 or 2 and R is a lower alkyl radical having 1 to 6 carbon atoms, or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R²⁴, R²⁵, R²⁶ and R²⁷ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R²⁴, R²⁵ or R²⁶, R²⁷ are an oxygen atom, or R²⁴, R²¹ or R²⁶, R²′, together with the carbon atom to which they are attached, form a saturated hydrocarbon ring having from 3 to 7 carbon atoms;

or an isomer or prodrug thereof.

Particular materials that are included in this family of compounds, and which can serve as the COX-2 inhibitor in the present invention, include N-(2-cyclohexyloxynitrophenyl)methane sulfonamide, and (E)-4-[(4-methylphenyl)(tetrahydro-2-oxo-3-furanylidene)methyl]benzenesulfonamide.

COX-2 inhibitors that are useful in the present invention include darbufelone (Pfizer), CS-502 (Sankyo), LAS 34475 (Almirall Profesfarma), LAS 34555 (Almirall Profesfarma), S-33516 (Servier, see Current Drugs Headline News, at http://www.current-drugs.com/NEWS/Inflaml.htm, Oct. 4, 2001), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis); BF-389 (Biofor/Scherer), GR-253035 (Glaxo Wellcome), 6-dioxo-9H-purin-8-yl-cinnamic acid (Glaxo Wellcome), and S-2474 (Shionogi).

COX-2 inhibitors that are useful in the invention can include the compounds that are described in U.S. Pat. Nos. 6,310,079; 6,306,890 and 6,303,628 (bicycliccarbonyl indoles); U.S. Pat. No. 6,300,363 (indole compounds); U.S. Pat. Nos. 6,297,282 and 6,004,948 (substituted derivatives of benzosulphonamides); U.S. Pat. Nos. 6,239,173, 6,169,188, 6,133,292; 6,020,343; 6,071,954; 5,981,576 ((methylsulfonyl)phenyl furanones); U.S. Pat. No. 6,083,969 (diarylcycloalkano and cycloalkeno pyrazoles); U.S. Pat. No. 6,222,048 (diaryl-2-(5H)-furanones; U.S. Pat. No. 6,077,869 (aryl phenylhydrazines); U.S. Pat. Nos. 6,071,936 and 6,001,843 (substituted pyridines); U.S. Pat. No. 6,307,047 (pyridazinone compounds); U.S. Pat. No. 6,140,515 (3-aryl-4-aryloxyfuran-5-ones); U.S. Pat. Nos. 6,204,387 and 6,127,545 (diaryl pyridines); U.S. Pat. No. 6,057,319 (3,4-diaryl-2-hydroxy-2,5-dihydrofurans; U.S. Pat. No. 6,046,236 (carbocyclic sulfonamides); and U.S. Pat. Nos. 6,002,014; 5,994,381; and 5,945,539 (oxazole derivatives).

In an embodiment of the present method, a subject in need of prevention, treatment or inhibition of a psychiatric disorder is treated with a COX-2 inhibitor or prodrug thereof. In particular the subject is treated with an effective amount of the COX-2 inhibitor, whereby the effective amount can be a therapeutic amount, and it can be an amount that is an effective amount for the prevention, treatment or inhibition of a psychiatric disorder.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of each agent for use in the combination therapy which will achieve the goal of improvement in the severity of neurological or psychiatric disorder and the frequency of incidence over treatment of each agent by itself, while avoiding adverse side effects typically associated with alternative therapies. Accordingly, a “therapeutic amount”, or a “therapeutically effective amount” of a medication is an amount that is therapeutically effective. Those skilled in the art will appreciate that dosages may also be determined with guidance from Goodman & Goldman's The Pharmacological Basis of Therapeutics, Ninth Edition (1996), Appendix II, pp. 1707-1711.

Preferred COX-2 inhibitors for the method of the present invention include celecoxib (Celebrex®), rofecoxib (Vioxx®), meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963, JTE-522, pharmaceutically acceptable salts, prodrugs or mixtures thereof. More preferred COX-2 inhibitors are celecoxib, parecoxib, valdecoxib, etoricoxib and rofecoxib.

According to a preferred embodiment of the method of the present invention, celecoxib (Celebrex®) or a pharmaceutically acceptable salt thereof is used. The term pharmaceutically acceptable salt includes salts that can be prepared according to known methods by those skilled in the art from the corresponding compound of the present invention, e.g. conventional metallic ion salts and organic salts.

Celecoxib can be administered at a dose of 50-1600 mg per day, preferably 200 to 600 mg, most preferably 400 mg per day. The administration can be carried out once or several times a day, preferably twice. The amount of celecoxib can be adapted depending on age, body weight and/or possible other diseases of the patient. Preferably, celecoxib is used in the form of tablets (Celebrex®) for oral administration.

Without intending to establish a certain theory as explanation for the observed effect of COX-2 inhibitors, the following mechanisms of action are taken into consideration.

There is no doubt that activation of COX-2 mediates inflammatory processes and that COX-2 is expressed in brain tissue. COX-2 can be activated by cytokines like IL-2, IL-6 and IL-10, and cytokine-activated COX-2 expression mediates further inflammatory processes. It was reported that IL-2 and soluble IL-2 receptors (Licino J, Seibyl, J P, Altemus M, Chamey D S, Krystal J H: Elevated levels of Interleukin-2 in neuroleptic-free schizophrenics. Am J Psychiatry 1993; 150: 1408-1410) (McAllister C G, van Kemmen D P, Rehn T J, Miller A L, Gurklis J, Kelley M E, Yao J, Peters J L: Increases in CSF levels of Interleukin-2 in schizophrenia: effects of recurrence of psychosis and medication status. Am J Psychiatry 1995; 152: 1291-1297), soluble IL-6 receptors as a functional part of the IL-6 system (Müller N, Dobmeier P, Empel M, Riedel M, Schwarz M, Ackenheil M: Soluble IL-6 Receptors in the serum and cerebrospinal fluid of paranoid schizophrenic patients. Eur Psychiatry 1997; 12: 294-299) and IL-10 (Van Kammen D P, McAllister-Sistilli C G, Kelley M E: Relationship between immune and behavioral measures in schizophrenia. In: G. Wieselmann (ed.) Current Update in Psychoimmunology, Springer Verlag 1997; Wien, NY, pp. 51-55) are increased in the cerebrospinal fluid of schizophrenic patients—the increase of the cytokines in the CNS may be accompanied by increased COX-2 expression. The effectiveness of COX-2 inhibitors, such as celecoxib, in the treatment of schizophrenia, might be based on the finding that celecoxib down-regulates the cytokine-induced CNS COX-2 activation.

Moreover, COX-2 inhibition seems to regulate the expression of adhesion molecules (Schwarz M J, Ackenheil M, Riedel M, Müller N: Blood-CSF-Barrier impairment as indicator for an immune process in schizophrenia. Neurosci Letters 1998; 253: 201-203). Since adhesion molecule regulation is impaired in schizophrenia, leading to dysbalance and lack of communication between the peripheral and the CNS immune system, the effects of COX-2 inhibitors, such as celecoxib, in the treatment of schizophrenia, may also be related to the adhesion molecules ICAM-1 and VCAM-1, especially regarding the negative symptoms (Schwarz M J, Riedel M, Gruber R, Ackenheil M, Müller N: Levels of soluble adhesion molecules in schizophrenia: Relation to psychopathology. In: N. Müller (Hrg) Psychiatry, Psychoneuroimmunology, and Viruses. Springer Verlag Wien, 1999, NY, pp. 121-130; Müller N, Ackenheil M: Immunoglobulin and albumin contents of cerebrospinal fluid in schizophrenic patients: The relationship to negative sympomatology. Schizophrenia Res 1995; 14: 223-228).

According to a further embodiment of the present invention, a method and composition for the prevention, treatment or inhibition of psychiatric disorders comprising administering COX-2 inhibitor in combination with a neuroleptic drug or an antidepressant are provided. The psychiatric disorders include schizophrenia, delusional disorders, affective disorders, autism and tic disorders, in particular chronic schizophrenic psychoses and schizoaffective psychoses, temporary acute psychotic disorders, depressive episodes, recurring depressive episodes, manic episodes and bipolar affective disorders. Combinations can also include a mixture of one or more COX-2 inhibitors with one or more neuroleptic agents or antidepressant. In particular, the combination of a COX-2 inhibitor with a neuroleptic drug is useful for the treatment of schizophrenia, whereas the combination of a COX-2 inhibitor with an antidepressant is applicable for the treatment of depressive disorders.

Both classical and atypical neuroleptics can be used for the add-on use according to the invention, atypical neuroleptics being preferred.

Examples of neuroleptic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table 3.

TABLE 3
Neuroleptic drugs

| | | | | Dosage |
|---|---|---|---|---|
| | | Route of | | Range and |
| Common Name | Trade Name | Administration | Form | (Median)a |
| Clozapine | Clozaril | oral | tablets | 12.5-900 mg/day |
| | | | | (300-900 mg/day) |
| Olanzapine | Zyprexa | oral | tablets | 5-25 mg/day |
| | | | | (10-25 mg/day) |
| Ziprasidone | Geodon | oral | capsules | 20-80 mg/twice a |
| | | | | day |
| | | | | (80-160 mg/day) |
| Risperidone | Risperdal | oral | solution | 2-16 mg/day |
| | | | tablets | (4-12 mg/day) |
| Quetiapine | Seroquel | oral | tablets | 50-900 mg/day |
| fumarate | | | | (300-900 mg/day) |
| Sertindole | SERLECT | | | (4-24 mg/day) |
| Amisulpride | | | | |
| Haloperidol | Haldol | oral | tablets | 1-100 mg/day |
| | | | | (1-15 mg/day) |
| Haloperidol | HALDOL | parenteral | injection | |
| Decanoate | decanoate | | | |
| Haloperidollactate | haldol | oral | solution | |
| | intensol | | | |
| | | parenteral | injection | |
| Chlorpromazine | Thorazine | rectal | suppositories | 30-800 mg/day |
| | | | | (200-500 mg/day) |
| | | oral | capsules | |
| | | | solution | |
| | | | tablets | |
| | | parenteral | injection | |
| Fluphenazine | Prolixin | | | 0.5-40 mg/day |
| | | | | (1-5 mg/day) |
| Fluphenazine | prolixin | parenteral | injection | (about one-half the |
| decanoate | decanoate | | | dosage shown for |
| | | | | oral) |
| Fluphenazine | prolixin | parenteral | injection | (same as above) |
| enanthate | | | | |
| Fluphenazine | prolixin | oral | elixer | |
| hydrochloride | | | solution | |
| | | | tablets | |
| | | parenteral | injection | |
| Thiothixene | Navane | oral | capsules | 6-60 mg/day |
| | | | | (8-30 mg/day) |
| Thiothixene | NAVANE | oral | solution | |
| hydrochloride | | | | |
| | | parenteral | injection | |
| Trifluoperazine | Stelazine | | | (2-40 mg/day) |
| Perphenazine | Trilafon | oral | solution | 12-64 mg/day |
| | | | tablets | (16-64 mg/day) |
| | | parenteral | injection | |
| Perpehazine and | ETRAFON | oral | tablets | |
| Amitriptyline | triavil | | | |
| hydrochloride | | | | |
| Thioridazine | Mellaril | oral | suspension | 150-800 mg/day |
| | | | solution | (100-300 mg/day) |
| | | | tablets | |
| Mesoridazine | | | | (30-400 mg/day) |
| Molindone | Moban | | | 50-225 mg/day |
| | | | | (15-150 mg/day) |
| Molindone | Moban | oral | solution | |
| hydrochloride | | | | |
| Loxapine | Loxitane | | | 20-250 mg/day |
| | | | | (60-100 mg/day) |
| Loxapine | loxitane | oral | solution | |
| hydrochloride | | | | |
| | | parenteral | injection | |
| Loxapine succinate | loxitane | oral | capsules | |
| Pimozide | | | | (1-10 mg/day) |
| Flupenthixol | | | | |
| Promazine | SPARINE | | | |
| Triflupromazine | VESPRIN | | | |
| Chlorprothixene | TARACTAN | | | |
| Droperidol | INAPSINE | | | |
| Acetophenazine | TINDAL | | | |
| Prochlorperazine | COMPAZINE | | | |
| Methotrimeprazine | NOZINAN | | | |
| Pipotiazine | PIPOTRIL | | | |
| Ziprasidone | | | | |
| Hoperidone | | | | |
| Zuclopenthixol | | | | |

Examples of tradenames and suppliers of selected neuroleptic drugs are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from SmithKlein Beckman); perphenazine (available under the tradename TRILAFON®, from Schering); thioridazine (available under the tradename MELLARIL®, from Novartis, Roxane, Hi-Tech, Teva, and Alpharma); molindone (available under the tradename MOBAN®, from Endo); and loxapine (available under the tradename LOXITANE® from Watson). Furthermore, benperidol (Glianimong), perazine (Taxilang) or melperone (Eunerpan®) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), ziprasidone, and hoperidone.

Preferred neuroleptic drugs include risperidone and aripiprazole (from Bristol Myers Squibb Company, see e.g. Stahl S M; Dopamine-system stabilizers, aripiprazole and the next generation of antipsychotics, part I, “goldilocks”-actions at dopamine receptors; J. Clin. Psychiatry 2001, 62, 11:841-842).

The most preferred neuroleptic drug within the present invention is risperidone (Risperdal®), its manufacture and pharmacological activity is described in EP 0 196 132. Risperidone acts as an antagonist to neurotransmitters, in particular dopamine, and is used for the treatment of psychoses.

Various types of antidepressants can be used for the add-on use according to the present invention. Examples of antidepressants that are useful in the present invention include, but are not limited to: tricyclic antidepressants such as amitriptyline (5-(3-dimethylamino propylidene)-10,11-dihydro-5H-dibenzo[a,d]cyclohepten), amitriptyline oxide, desipramine (10,11-dihydro-5-(3-methylamino propyl)-5H-dibenz[b,j]azepin), dibenzepin (10-(2-dimethylamino ethyl)-5,11-dihydro-5-methyl-1H-dibenzo[b,e][1,4]diazepin-11-on), dosulepin (3-(6H-dibenzo[b, e]thiepin-11-yliden)-N,N-dimethylpropyl amine), doxepin (3-(6H-dibenz[b,e]oxepin-11-yliden)-dimethylpropyl amine), chloroimipramine, imipramine (5-(3-dimethylamino propyl)-5,11-dihydro-5H-dibenz[b,f]azepin), nortriptyline (3-(10,11-dihydro-5H-dibenzo[a,d]cyclohepten-5-yliden)-N-methyl-1-propane amine), mianserin (1,2,3,4,10,14b-hexahydro-2-methyl-dibenzo[c,f]pyrazino[1,2-a]azepin), maprotiline (N-methyl-9,10-ethanoanthracene-9(10H)-propane amine), trimipramine (5-[3-dimethylamino)-2-methylpropyl]-10,11-dihydro-5H-dibenz[b,f]-azepin) or viloxazine (RS)-2-(2-ethyoxy phenoxy methyl)-morpholine), modern antidepressants such as trazodone (2-{3-[4-(3-chlorophenyl)-1-piperazinyl]-propyl}-1,2,4-triazolo[4,3-a]pyridine-3(2H)-on, nefazodone (2-{3-[4-(3-chlorophenyl)-1-piperazinyl]propyl}-5-ethyl-2,4-dihydro-4-(2-phenoxyethyl)-3H-1,2,4-triazol-3-on), mirtazapine ((±)-1,2,3,4,10,14b-hexahydro-2-methylpyrazino[2,1-a]pyrido[2,3-c][2]benzazepin), venlafaxine ((±)-1-2-(dimethylamino)-1-(4-methoxyphenyl)-ethyl]cyclohexanol) or reboxetine ((±)-(2RS)-2-[α(SR)-α-(2-ethoxyphenoxy)benzyl]morpholine), inhibitors of monoaminooxidases such as tranylcypromine (trans-2-phenyl cyclopropyl amine), brofaromine or moclobemide (4-chloro-N-(2-morpholinoethyl)-benzamide), selective inhibitors of serotonin-uptake such as citalopram, paroxetine, fluoxetine ((RS)-N-methyl-3-phenyl-3-[4-(trifluoromethyl)phenoxy]propyl amine, available under the tradename PROZAC®), fluvoxamine ((E)-5-methyoxy-4′-(trifluoromethyl)-valerophenon-O-(2-aminoethyl)oxime) or sertraline ((1S-cis)-(+)-4-(3,4-dichlorophenyl)-1,2,3,4-tetrahydro-N-methyl-1-naphthalinamine), and vegetable antidepressants such as Hypericum (St. John's wort).

In the present method, the subject is treated with an amount of a neuroleptic agent or antidepressant and an amount of a COX-2 inhibitor, where the amount of the neuroleptic agent or antidepressant and the amount of the COX-2 inhibitor together provide a dosage or amount of the combination that is sufficient to constitute an effective amount of the combination. The effective amount can be a therapeutic amount, and it can be an amount that is an effective amount for the prevention, treatment or inhibition of a psychiatric disorder. In the present method, the amount of the neuroleptic agent or antidepressant that is used is such that, when administered with the COX-2 inhibitor, it is sufficient to constitute an effective amount of the combination. It is preferred that the dosage amount of the neuroleptic agent or antidepressant and the dosage amount of the COX-2 inhibitor constitute a therapeutically effective amount of the combination of the two.

It is well known that different neuroleptic agents or antidepressant have different levels of potency and that recommended dosage levels vary considerably. The recommended dosage level for a commercial neuroleptic agent or antidepressant can be found in the prescribing information that is published by the distributor. Some allowable and preferred dosage levels for selected neuroleptic agents that are preferred for use in the present invention are shown in Table 3.

According to a preferred embodiment of present method and composition, the neuroleptic risperidone is administered at a dose of 2-6 mg/day, preferably 4-5 mg. The dose for celecoxib may range from 50-1600 mg/day, preferably 200-600, more preferably 400 mg. Preferably, the administration occurs twice daily (in the morning and in the evening).

The frequency of dose will depend upon the half-life of the neuroleptic agent or antidepressant molecule. If the molecule has a short half life (e.g. from about 2 to 10 hours) it may be necessary to give one or more doses per day. Alternatively, if the molecule has a long half-life (e.g. from about 2 to about 15 days) it may only be necessary to give a dosage once per day, per week, or even once every 1 or 2 months. A preferred dosage rate is to administer the dosage amounts described above to a subject once or twice per day. The amount of COX-2 selective inhibitor that is used in the subject method may be an amount that, when administered with the neuroleptic agent or antidepressant, is sufficient to constitute an effective amount of the combination. Preferably, such amount would be sufficient to provide a therapeutically effective amount of the combination.

In the present method, and in the subject compositions, the neuroleptic agent or antidepressant is administered with, or is combined with, a COX-2 inhibitor.

The combination of a neuroleptic agent and a COX-2 inhibitor, or of an antidepressant and a COX-2 inhibitor, can be supplied in the form of a novel therapeutic composition that is believed to be within the scope of the present invention. The relative amounts of each component in the therapeutic composition may be varied. The COX-2 inhibitors and the neuroleptic agents or antidepressants can be provided in the therapeutic composition so that the preferred amounts of each of the components are supplied by a single dosage, a single injection or a single capsule for example, or, by up to four, or more, single dosage forms.

Any one or more of the COX-2 inhibitors that are described above can be combined with any one or more of the neuroleptic agents are antidepressants that are described above in the novel method and combinations of the present invention. By way of example, combinations can include a COX-2 selective inhibitor, such as celecoxib, rofecoxib, parecoxib, valdecoxib, etoricoxib, deracoxib, and NS-398, and a neuroleptic agent, such as clozapine, olanzapine, ziprasidone, risperidone, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone and zuclopenthixol. Combinations can also include a mixture of one or more COX-2 selective inhibitors with one or more neuroleptic agents.

When the novel combination is supplied along with a pharmaceutically acceptable carrier, a pharmaceutical composition is formed. The pharmaceutical composition comprises a pharmaceutically acceptable carrier, a neuroleptic agent or antidepressant, and a COX-2 inhibitor. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The invention is also directed to a novel kit that is suitable for use in the treatment of psychiatric disorders such as schizophrenia, delusional disorders, affective disorders, autism or tic disorders, comprising a first dosage form comprising a neuroleptic agent or antidepressant and a second dosage form comprising a COX-2 inhibitor or prodrug thereof, in quantities which comprise a therapeutically effective amount of the combination of the compounds for the treatment, prevention, or inhibition of a psychiatric disorder, for simultaneous, separate or sequential administration.

According to a preferred embodiment, the dosage form comprising a neuroleptic agent or antidepressant and the second dosage form comprising a COX-2 inhibitor are administered simultaneously. Examples for COX-inhibitors and neuroleptics or antidepressants usable in the kit-of-parts of the present invention are listed above with reference to methods or compositions of the present invention.

The subject pharmaceutical kit may be administered enterally (orally) or parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. Preferably the administration of a pharmaceutical kit comprising a COX-2 inhibitor and a neuroleptic or antidepressant occurs enterally (orally), in form of tablets.

According to a preferred embodiment, the kit comprises celecoxib or a pharmaceutically acceptable salt thereof as COX-2 inhibitor and risperidone as neuroleptic drug. Most preferably, the celecoxib is comprised in an amount of 50-1600 mg, preferably 200-600 mg and most preferably 400 mg, and risperidone in an amount of 2-6 mg, preferably 4-5 mg.

The treatment of psychiatric disorders with COX-2 inhibitors, alone or in combination with a neuroleptic or antidepressant, may occur in addition to further drug therapies. Thus, tranquilizers may be used for the treatment of agitation, anxiety or sleep disturbances. Preferably lorazepam is used, which belongs to the class of benzodiazepines.

### EXAMPLES

In the following, the invention will be discussed in more detail with reference to a patient study. Other embodiments within the scope of the claims herein will be apparent to one skilled in the art from consideration of the specification or practice of the invention as disclosed herein. The results of the patient study are graphically represented in the attached figures.

### Example 1

The study was performed as a single-center, double-blind, placebo-controlled, randomized, parallel-groupe valuation of the combination therapy with celecoxib and risperidone versus a monotherapy with risperidone and placebo in schizophrenic patients. The study included 50 patients fulfilling the criteria for the diagnosis of schizophrenia according to DSM IV (American Psychiatric Association (1994), Diagnostic and Statistical Manual of Mental Disorders, 1st Edition, American Psychiatric Press, Washington D.C.), of whom 25 belonged to the risperidone-placebo and 25 to the risperidone-celecoxib group. No significant differences were present between the two patient groups were found with regard to age, sex, duration or severity of the disease or psychopathology, risperidone dose or risperidone-plasma levels.

The patients received 2-6 mg/day of risperidone (Risperdal®), and depending on to which group they belonged, 400 mg/day of celecoxib (2×200 mg Celebrex® mornings and evenings) or placebo over 5 weeks after a brief wash-out period of earlier antipsychotic medication. During the wash-out period, a benzodiazepine preparation (mostly lorazepam) was prescribed, if necessary. Patients with agitation, anxiety, or sleeping problems were also medicated with lorazepam during the study.

The psychopathology of the patients was assessed using the positive and negative syndrome scale (PANSS) (Kay et al., Schizophr. Bull. 1987, 13:261-276).

The extrapyramidal side effects were assessed by the EPS scale (Simpson and Angus, Acta Psychiat. Scand. 1970 (Suppl.), 212). The use of biperiden was monitored as a possible indicator for side effects of the antipsychotic medication.

In order to exclude the chance that possible differences in the therapeutic effectiveness between the two groups might be due to non-compliance during the risperidone therapy or to differences in risperidone metabolism, the plasma levels of risperidone or 9-OH-risperidone were monitored during the study.

The statistics were performed according to the criterion of “last observation carried forward” (LOCF), i.e., the last PANSS scores of the patients who dropped out before the end of the study were carried forward to all subsequent observation days. For the comparison of the main efficacy parameter, the mean change in the PANSS between the two treatment groups, t-tests for independent samples were employed. With reference to the underlying hypothesis of a better outcome of the celecoxib-risperidone group, a significance of p<0.05 was calculated in the one-tailed t-test and used as the basis for the estimation of the sample size (statistical power) and for the comparison of the groups. For all other comparisons, two-tailed t-tests were used.

At the start of the study, in the risperidone-celecoxib group (average age 35.9±12.8 years), the PANSS total score was 71.8±17.1, the PANSS global score was 34.0±8.5, the PANSS positive score was 19.0±5.9 and the PANSS negative score was 18.7±6.3. In the risperidone-placebo group (average age 35.5±13.6 years), the PANSS total score was 75.4±12.9, the PANSS global score was 37.2±7.1, the PANSS positive score was 17.2±4.6 and the PANSS negative score was 21.1±5.5. Consequently, there was no significant difference in the PANSS total score or any of the subscales.

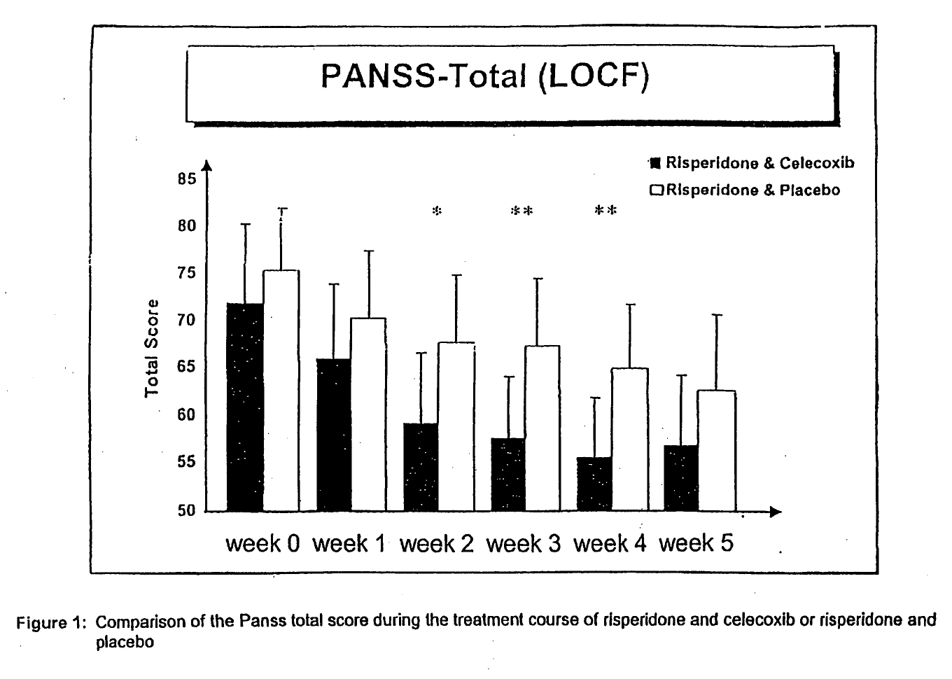

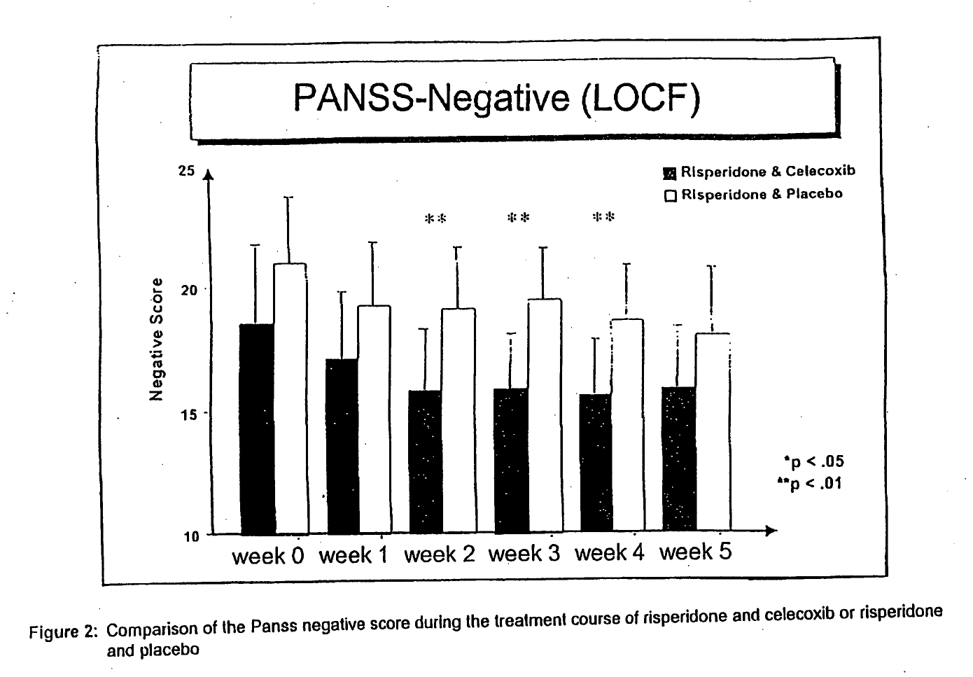

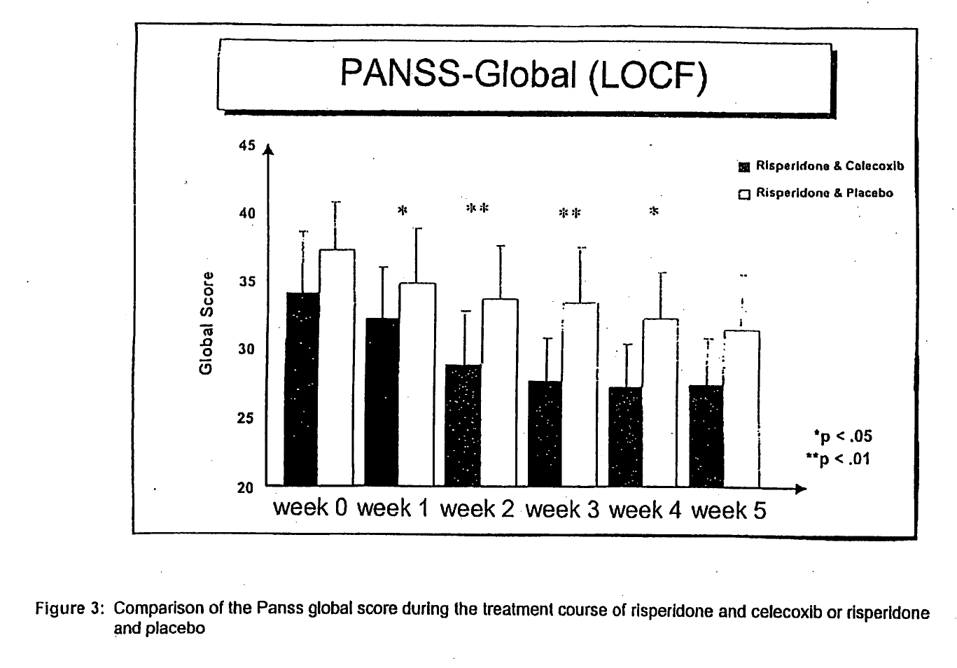

During the five-week therapy, a significant improvement of the PANSS total score and the subscales is observed in both groups of schizophrenic patients. The results of the PANSS total score are shown in FIG. 1, of the PANSS negative score in FIG. 2, of the PANSS global score in FIG. 3 and of the PANSS positive score in Table 4.

TABLE 4
Comparison of the PANSS positive score

| | Celecoxib and | Placebo and | | |
|---|---|---|---|---|
| Time | Risperidone | Risperidone | t1 | P2 |
| week 0 | 19.0 ± 5.9 | 17.2 ± 4.6 | 1.22 | n.s.3 |
| week 1 | 16.7 ± 5,5 | 16.2 ± 4.6 | 0.36 | n.s. |
| week 2 | 14.4 ± 5.0 | 15 ± 4.5 | 0.42 | n.s. |
| week 3 | 14.0 ± 4.7 | 14.5 ± 4.6 | 0.36 | n.s. |
| week 4 | 12.8 ± 4.4 | 14.2 ± 4.4 | 1.16 | n.s. |
| week 5 | 13.4 ± 5.6 | 13.3 ± 4.4 | 0.11 | n.s. |

1 t represents the statistical random sample distribution.
2 p represents the statistical power (probability).
3 n.s. means no statistical significance.

In the celecoxib-risperidone group, the two-tailed t-tests between the baseline and week 5 gave the following values: PANSS total score p<0.0001, PANSS global score p<0.0001, PANSS positive score p<0.0001, PANSS negative score p<0.001. In the placebo-risperidone group, the t-tests between the baseline and week 5 gave the following values: PANSS total score p<0.002, PANSS global score p<0.003, PANSS positive score p<0.002, PANSS negative score p<0.02.

The improved effectiveness of the combination therapy with celecoxib-risperidone in comparison to risperidone monotherapy is clearly shown by the significantly lower PANSS global scores after the 2, 3, 4 and 5 weeks of treatment (FIG. 3). With regard to the total and negative score, significantly lower scores were recorded after 2, 3 and 4 weeks in the celecoxib-risperidone group (FIGS. 1 and 2).

The mean daily dose of risperidone is shown in Table 5; no statistically significant difference was found between the two treatment groups.

TABLE 5
Mean risperidone dose mg/day

| | Celecoxib and | Placebo and | |
|---|---|---|---|
| Time | Risperidone | Risperidone | Difference |
| week 1 | 4.1 ± 0.6 | 4.0 ± 0.8 | n.s. |
| week 2 | 4.5 ± 0.6 | 4.4 ± 1.1 | n.s. |
| week 3 | 4.8 ± 0.8 | 4.9 ± 1.4 | n.s. |
| week 4 | 5.0 ± 1.0 | 4.9 ± 1.4 | n.s. |
| week 5 | 4.9 ± 1.0 | 5.1 ± 1.5 | n.s. |

1 n.s. means no statistical significance.

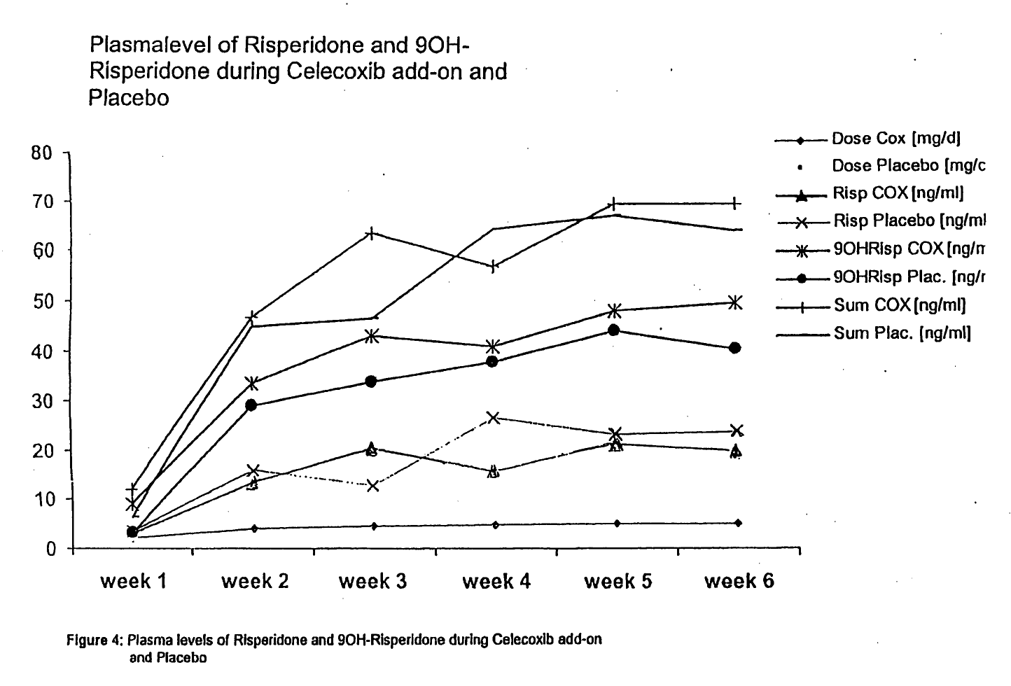

The differences in the plasma levels of risperidone or the metabolite 9-OH-risperidone shown in FIG. 4 were also without statistical significance (the present FIG. 4 differs from FIG. 4 of the German patent application priority document due to a calculation error in said priority document).

Therefore, it could be excluded that the observed differences in the therapeutic effectiveness between the two groups are due to incompatibility during the risperidone therapy or differences in risperidone metabolism. The therapeutic benefit of the combined therapy has to be attributed to the COX-2 inhibitor, celecoxib.

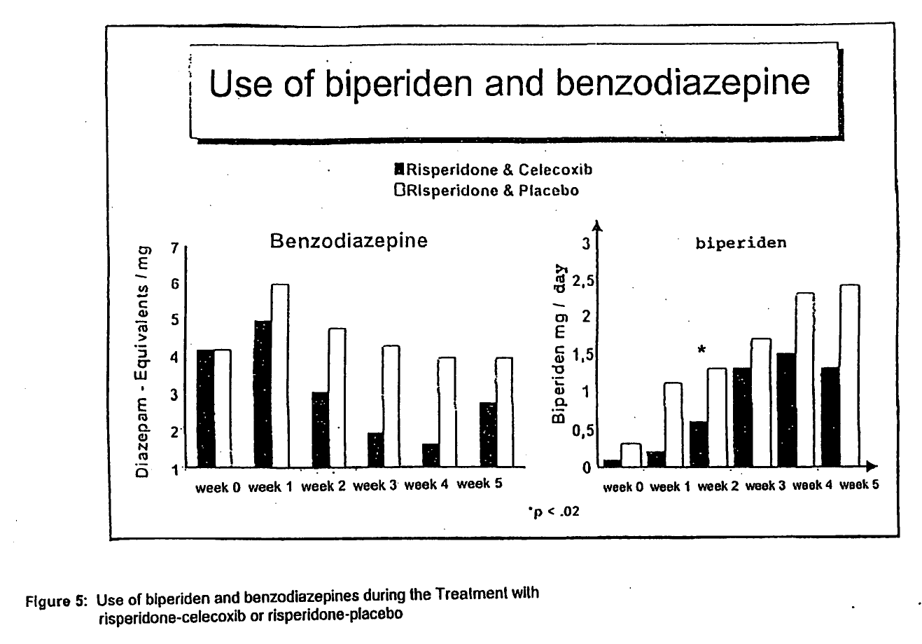

With respect to the extrapyramidal side effects, no statistically significant differences were found in the EPS scale. The use of biperiden is shown in FIG. 5 and was calculated as cumulative weekly dose. The values were lower in the celecoxib-risperidone group, and reached statistical significance at week 2 (p<0.02).

A detailed analysis of items of the PANSS-Scale which discriminate good celecoxib-responders from the placebo group revealed that therapeutic effects of celecoxib are especially found on the items “lack of contact” (item 3 of the negative subscale), “emotional isolation” (item 2 of the negative subscale), “passive-apathic isolation” (item 4 of the negative subscale), “social withdrawal” (item 16 of the general psychopathology subscale), “depression” (item 6 of the general psychopathology subscale) and “motor retardation” (item 6 of the general psychopathology subscale).

Furthermore, a factor analysis showed that especially items which can subsumed under the label “agitation” show a good therapeutic response to celecoxib, but not to placebo. All those items reflect psychopathological symptoms which are typically found in depressive states. Therefore this detailed analysis points to a therapeutic efficiency in depressive states.

Moreover, “passive-apathic isolation”, “motor retardation”, “social withdrawal”, or “lack of contact” are—often more severe expressed than in depressive states—also core-symptoms of childhood autism.

The combination of celecoxib and risperidone according to the present invention thus shows improved results compared to the monopreparation risperidone with regard to effectiveness in the treatment of schizophrenia. Furthermore, it was observed that the beneficial effects of the add-on therapy occurred faster in patients with a recent onset of the disorder and that the celecoxib therapy was useful in the treatment of depressive states.

### Example 2

### The COX-2 Inhibitor Rofecoxib Possesses Antidepressant Activity

Various behavioural test paradigms have been developed for evaluating the antidepressant properties of novel drugs in animals. One of the most reliable and specific paradigm is the forced swim test which has been successfully used to determine the effectiveness of antidepressants, evaluate new pharmaceutical compounds and validate animal models of depression (Porsolt et al. (1977) Arch. Int. Pharmacodym. 229:327-336; Porsolt (2000) Rev. Neurosci. 1 1:53-58; Rénéric et al. (2002) Behav. Brain Res. 136:521-532; Page et al. (2003) Psychopharmacology 165:194-201; Kelliher et al. (2003) Psychoneuroendocrinology 28:332-347). The test consists of placing a mouse for a period of 5 minutes into a glass cylinder containing a water depth of at least 15 cm. Under such circumstances, a mouse cannot touch the bottom of the cylinder and is thus forced to swim. Time, latency and frequency of struggling/swimming versus floating are scored as behavioural parameters. Floating (i.e. movements made only for keeping balance and breath) can be interpreted as a depression-like behaviour that reflects either a failure of persistent escape-directed behaviour (i.e. behavioural despair) or the development of passive behaviour that disengages the animal from active forms of coping with stress stimuli. By contrast, increased struggling (i.e. vigorous attempts to escape) and swimming indicates active coping behaviour and can be interpreted as an opposite to depression-like behaviour. Treatment with existing antidepressants has been shown to reduce the total time spent floating while increasing the time spent swimming and/or struggling, which is interpreted as an improvement in depression-like behaviour (Rénéric et al. (2002) Behav. Brain Res. 136:521-532; Page et al. (2003) Psychopharmacology 165:194-201; Kelliher et al. (2003) Psychoneuroendocrinology 28:332-347).

The antidepressant activity of rofecoxib was assessed in animals according to the forced swim test. Briefly, rofecoxib was given orally by gavage to mice either in a single acute administration of 10 mg or in a repetitive chronic administration of 4 mg per day (2 mg at 9:00 in the morning, 2 mg at 18:00 in the evening) for 28 days. Control animals received a negative control consisting of water only. The forced swim test was performed 4 hour after the last administration of rofecoxib. All experiments were performed using a selected DBA/2Ola mouse strain that displays characteristics of being anxious and responds to antidepressant treatment. All observed results were confirmed statistically using the one-way ANOVA test.

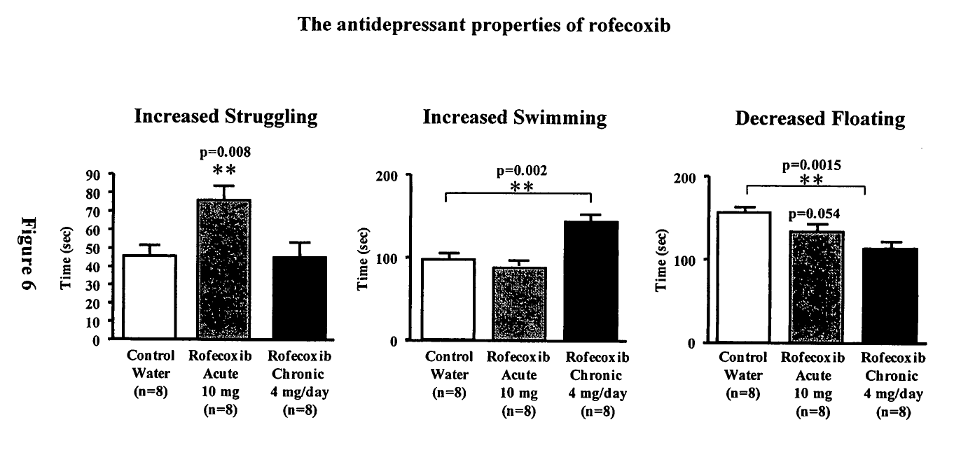

As shown in FIG. 6, acute and chronic administration of rofecoxib statistically increased either struggling or swimming times while decreasing floating time. These results demonstrate that rofecoxib increases stress coping behaviour, which is interpreted as an improvement of depression-like behaviour.

### Example 3

### The COX-2 Inhibitor Valdecoxib Possesses Antidepressant Activity

The antidepressant activity of valdecoxib was assessed in DBA/2Ola mice according to the forced swim test. Briefly, chronic administration of valdecoxib was performed by oral gavage for 28 days at a concentration of 4 mg per day (2 mg at 9:00 in the morning, 2 mg at 18:00 in the evening) and 20 mg per day (10 mg at 9:00 in the morning, 10 mg at 18:00 in the evening). Control animals received a placebo consisting of water only. The behaviour of individual animals was assessed using the forced swim test 24 hours after the last administration of valdecoxib. A pre-exposure of 5 minutes to the test was done 4 hours after the last administration of valdecoxib. All observed results were confirmed statistically using the one-way ANOVA test.

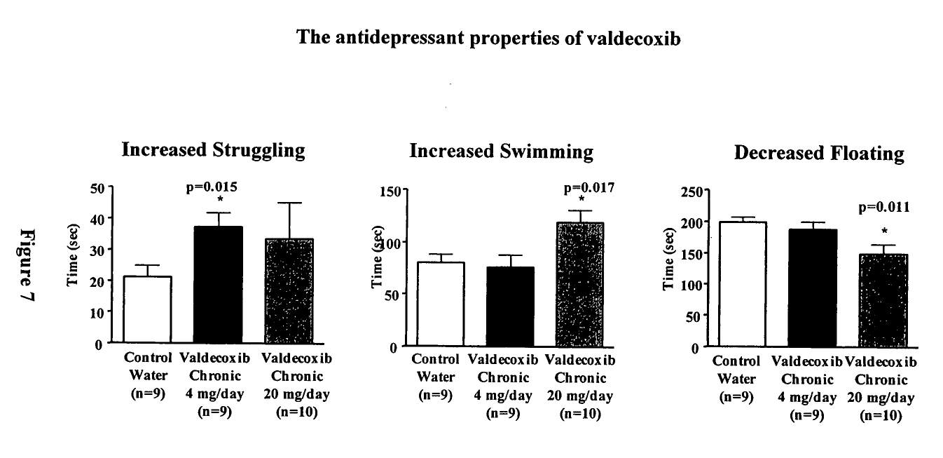

As shown in FIG. 7, chronic administration of valdecoxib statistically increased either struggling or swimming times while decreasing floating time. These results demonstrate that valdecoxib increases stress coping behaviour, which is interpreted as an improvement of depression-like behaviour.

### Example 4

### The COX-2 Inhibitor Etoricoxib Possesses Antidepressant Activity

The antidepressant activity of etoricoxib was assessed in DBA/2Ola mice according to the forced swim test. Briefly, etoricoxib was chronically administered to mice by oral gavage for 28 days at a concentration of 4 mg per day (2 mg at 9:00 in the morning, 2 mg at 18:00 in the evening) and 20 mg per day (10 mg at 9:00 in the morning, 10 mg at 18:00 in the evening). Control animals received a placebo consisting of water only. The behaviour of individual animals was assessed using the forced swim test 24 hours after the last administration of etoricoxib. A pre-exposure of 5 minutes to the test was done 4 hours after the last administration of etoricoxib. All observed results were confirmed statistically using the one-way ANOVA test.

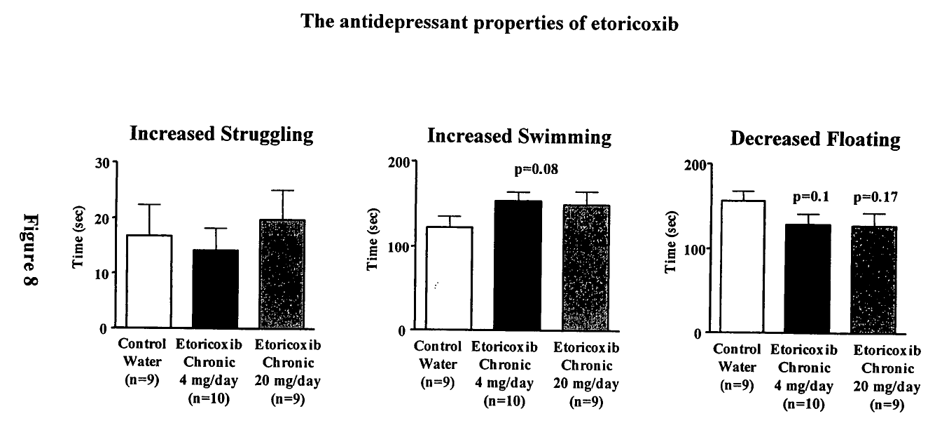

As shown in FIG. 8, chronic administration of etoricoxib statistically increased or showed a tendency to increase either struggling or swimming times while decreasing floating time. These results demonstrate that etoricoxib increases stress coping behaviour, which is interpreted as an improvement of depression-like behaviour.

### Example 5

### The COX-2 Inhibitor Piroxicam Possesses Antidepressant Activity

The antidepressant activity of piroxicam was assessed in DBA/2Ola mice according to the forced swim test. Briefly, chronic administration of piroxicam was performed by oral gavage to mice for 28 days at a concentration of 5 mg per day (2.5 mg at 9:00 in the morning, 2.5 mg at 18:00 in the evening). Control animals received a placebo consisting of water only. The behaviour of individual animals was assessed using the forced swim test 24 hours after the last administration of piroxicam. A pre-exposure of 5 minutes to the test was done 4 hours the last administration of piroxicam. All observed results were confirmed statistically using the one-way ANOVA test.

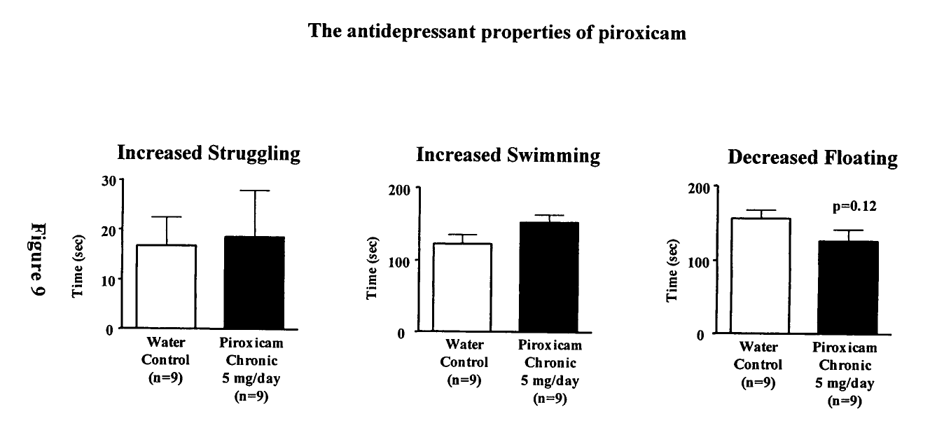

As shown in FIG. 9, chronic administration of piroxicam resulted in a tendency to increase either struggling or swimming times while decreasing floating time. These results demonstrate that piroxicam increases stress coping behaviour, which is interpreted as an improvement of depression-like behaviour.

### Example 6

### The COX-2 Inhibitor Celecoxib Possesses Antidepressant Activity

The antidepressant activity of celecoxib was demonstrated in a clinical study involving 40 depressed patients.

The study was conducted as a double-blind, randomized, placebo controlled, prospective parallel group trial of celecoxib add-on to reboxetine. The treatment period lasted 42 days (6 weeks) after a wash-out period of at least three days in pre-medicated patients. All patients suffered from MD (DSM IV: 296.2× single depressive episode or 296.3× recurrent depressive episode). 40 patients (20 f, 20 m) aged between 23 and 63 years were included in the study. 37 of the patients included were in-patients. 12 males and 8 females were included in the celecoxib-group and 8 males and 12 females in the placebo-group. 34 patients were included in Munich and six patients in Münster. Patients suffering from psychotic depression were excluded. Each patient was included after written informed consent. The study was examined by the ethics committee of the medical faculty of the University of Munich.

The psychopathology of the patients was assessed by raters, who had undergone a training program, using the Hamilton-Depression scale, 17—item version (HamD), 24. Assessment of psychopathology and other examinations were performed at weekly intervals. At baseline, no difference could be seen between the groups regarding the severity of depression.

During the wash-out and the treatment periods the patients additionally received the benzodiazepine lorazepam for acute agitation or anxiety.

Celecoxib and placebo were administered in identical capsules produced by the pharmacy of the medical faculty Munich according to the randomization scheme. The dose of reboxetine was flexible and ranged from 4 mg/day to 10 mg/day, according to clinical needs. Celecoxib was administered at a dose of 400 mg/day. Reboxetine was started with 2 mg for two days before administering 4 mg, celecoxib was titrated from 200 mg/day to 400 mg/day within three days.

In order to exclude the chance that any differences in treatment response between the groups might be due to noncompliance during reboxetine therapy or to differences in reboxetinemetabolism (e.g. through reboxetine—celecoxib interactions), reboxetine plasma levels were monitored during the study. An overview on the characteristics of the patients and doses of drugs is shown in Table 6.

TABLE 6
Overview on characteristics of patients receiving celecoxib or placebo
(mean ± SD)

| | celecoxib | placebo |
|---|---|---|
| | (n = 20) | (n- = 20) |
| sex | 12 m, 8 f | 8 m, 12 f |
| age range | 25-63 years | 23-65 years |
| mean age | 44.5 ± 11.6 years | 44.3 ± 13.5 years |
| age of onset | 36.2 ± 12.4 years | 37.5 ± 15.0 years |
| episodes | | |
| (including present) | | |
| range | 1-11 | 1-5 |
| mean | 2.5 ± 2.3 | 2.4 ± 1.2 |
| hospitalizations | | |
| (incl. pres.) | | |
| range | 1-11 | 0-5 |
| mean | 1.1 ± 0.3 | 1.6 ± 1.2 |
| duration present episode | 17.0 ± 21.7 | 18.7 ± 20.8 |
| (weeks) | | |
| reboxetine dose | | |
| range | 4-10 mg/day | 4-10 mg/day |
| mean | 6.79 ± 0.82 mg/day | 6.81 ± 1.14 mg/day |
| benzodiazepine dose | | |
| (diazepam-equivalents) | | |
| range | 0-7 mg/day | 0-10 mg/day |
| Mean | 2.4 mg ± 3.0 mg/day | 2.7 mg ± 3.1 mg/day |

At inclusion into the study the severity of depression ranged from 15 to 38 points on the HamD scale. The drop-out rate was relatively high in both groups. 10 patients dropped out from the celecoxib group before the trial end. Five of them were excluded or refused further treatment in the study due to a lack of therapeutic efficacy, four patients were excluded due to side-effects of the treatment (increase in blood-pressure, sleep-disturbance, difficulties in miction or erection, exanthema of the skin). Regarding the point of time for drop-out, patients from the celecoxib-group dropped out later: three patients after three weeks, five after four weeks and two during the last week of the trial. Of the latter two, one patient refused the last visit because he was discharged from the hospital and felt healthy.

From the placebo-group, twelve patients dropped before the end of the study. Nine of them were excluded or denied further treatment due to a lack of therapeutic efficacy, three patients were excluded due to side-effects of the treatment (nausea, agitation, sinus-tachychardia). Three patients dropped-out already after only two weeks, four after three weeks and five after four weeks.

In the celecoxib group, no cardiovascular events or side-effects were observed, neither clinically nor by ECG surveillance.

For statistics, analysis of variance was used for the HamD scale. The degrees of freedom for the within-subjects comparisons were corrected for deviance from sphericity (Greenhouse-Geisser). Post-hoc t tests were used for the weekly comparison of HamD scores. An intent to treat analysis was performed using the “last observation carried forward” (LOCF) procedure. For the comparison of reboxetine plasma levels, the pair-wise t test was used.

A statistically significant decrease of the depressive symptoms was observed in both treatment groups during the trial on the HamD scores (HamD testing over time: Greenhouse-Geisser-corrected F=36,776; df 2.452; p<0.0001). The effects of reboxetine treatment, however, was not the focus of our study. The decrease, however, was much greater in the group who received the add-on medication of celecoxib (testing time * group: GreenhouseGeisser-corrected F=3.220; df 2.434; p=0.035).

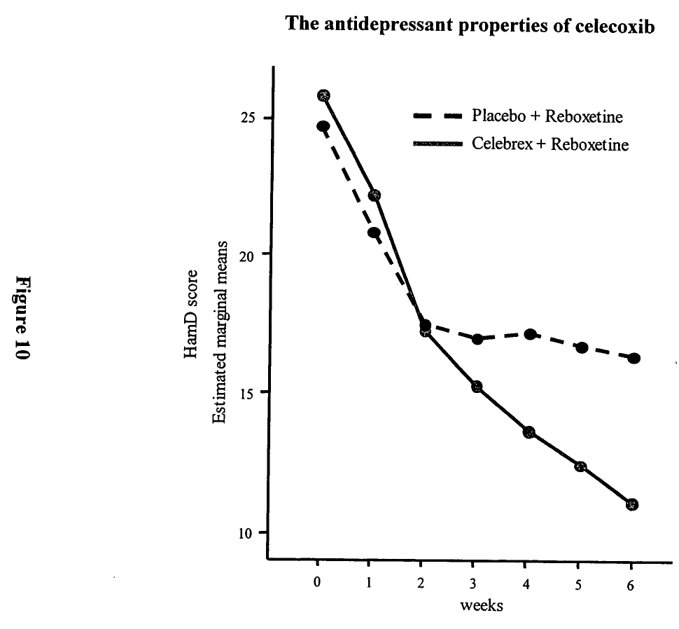

The course of the HamD scores is shown in FIG. 10 (LOCF), the course of the HAMD scores of the completers of the study in Table 7.

TABLE 7
Course of the HAMD scores during therapy with celecoxib or placebo,
only completers (mean ± SD)

| | Reboxetine and Celecoxib | Reboxetine and placebo |
|---|---|---|
| before treatment | 25.4 ± 4.0 | 24.5 ± 5.9 |
| week 1 | 19.8 ± 5.5 | 20.5 ± 7.8 |
| week 2 | 15.8 ± 5.0 | 17.3 ± 7.6 |
| week 3 | 13.9 ± 6.5 | 16.4 ± 6.8 |
| week 4 | 12.9 ± 7.0 | 16.9 ± 8.7 |
| week 5 | 10.1 ± 6.6 | 13.3 ± 9.3 |
| week 6 | 7.9 ± 7.1 | 12.1 ± 8.3 |

The mean decrease of the depressive symptoms between baseline and the end of the trial was 14 HamD score points or 55% in the reboxetine and celecoxib group and 8.1 points or 33% in the reboxetine and placebo group according to the LOCF criterion. The completers only showed an improvement of 17.5 scores in the celecoxib-group and of 12.4 in the placebo group, i.e. 69% improvement in the celecoxib group and 49% in the placebo group.

Side effects that have been attributed to the administration of celecoxib, especially gastrointestinal problems, were not observed. One patient who was receiving reboxetine and placebo dropped out of the study because of nausea. The reasons leading to study drop-out in the celecoxib (and in the reboxetine-) group are described in medical literature as typical effects of noradrenergic drugs such as reboxetine.

No statistical significant difference could be observed between both groups regarding the plasma levels of reboxetine (see Table 8) or the use of benzodiazepines (see Table 6).

TABLE 8
Plasma levels of reboxetine over 6 weeks for patients with major
depression treated with reboxetine plus celecoxib or reboxetine
plus placebo

| | reboxetine plus | | reboxetine plus | | | |
|---|---|---|---|---|---|---|
| | celecoxib (ng/ml) | | placebo (ng/ml) | | | |
| | Mean | SD | Mean | SD | t | p |
| Week 1 | 211.2 | 103.0 | 191.4 | 85.9 | .38 | 0.56 |
| Week 2 | 229.1 | 122.0 | 227.5 | 127.3 | .04 | 0.97 |
| Week 3 | 230.4 | 115.6 | 270.6 | 166.8 | .74 | 0.46 |
| Week 4 | 246.4 | 124.7 | 322.1 | 207.4 | 1.13 | 0.30 |
| Week 5 | 228.2 | 110.1 | 264.5 | 117.5 | .71 | 0.48 |
| Week 6 | 308.0 | 197.1 | 198.3 | 121.0 | 1.49 | 0.17 |

These results demonstrate that COX-2 inhibitors such as celecoxib can be used to treat depression.

### Example 7

### COX-2 Inhibitors can be Used to Treat Tic Disorders

In order to assess the therapeutic potential of COX-2 inhibitors in the treatment of ticdisorders, the COX-2 inhibitor celecoxib was administered to a patient suffering of ticdisorders.

Mr. A., a 23-years old man suffered from motor and vocal tics since the age of 11. Motor tics included simple tics such as shaking the arms and the head, complex motor tics touching other people and spitting. Vocal tics included throat-cleaning, palilalia, swearing obscenities and just right phenomena such as repeating sentences until they are “formulated in a right way”. He had a loss of impulse control hurting himself and showing aggressive behaviour or verbal attacks against other people. A suppression of the tics and aggression was possible sometimes for hours. As a consequence of his behaviour, he was socially withdrawn and had contact only to close family members, he did not leave the house and stopped further education after high-school. The symptoms were chronic, not waxing and waning over the last five years. The mood was partly ashamed and depressed due to his behaviour. He had difficulties to concentrate and to take attention.

Over the years he was medicated with haloperidole, pimozide, risperidone, amisulpride, tiapride, carbamazepine, and propanolol. Haloperidol had advantageous effects on the tics in higher doses, but he suffered from extrapyramidal side-effects and did not tolerate further treatment with this substance. Pimozide 12 mg or other treatment regimes had not a sufficient effect.

The blood-tests revealed slight positive serological titers for anti-DNase (660 IU/ml; normal <250 IU/ml) and for mycoplasma pneumoniae 1 gM (14 IU/ml; normal <13 IU/ml).

Antibiotic therapy with azithromycin 500 mg once a week was performed over 10 weeks. Three months later spitting and touching were improved and the patient was socially less withdrawn. Due to the persisting, although improved, tics and the impulsive and aggressive behaviour, first zotepine and later ziprasidone were added to the antibiotic prophylaxis using 1200 mg azithromycin every two weeks, which was started after about half a year without medication. Zotepine was not tolerated due to sedation, ziprasidone had no clear-cut effect on tics or behavioural disturbances.

Due to the persisting symptoms and the consideration that the symptoms might be result of an inflammatory process in the CNS, 2×200 mg celecoxib was added to the continuing antibiotic prophylaxis. During the next months, a further improvement of motor and vocal tics, the aggressive behaviour, and the social withdrawal could be observed. The patient started to work in the company of his father, he went out with friends, and the concentration improved.

In order to separate possible effects of the antibiotic therapy from the COX-2 inhibitor, celecoxib was withdrawn after about six months. Within two weeks, a worsening in cognitive function and patience was observed. The mood became more depressed on the one hand, impulsive and aggressive on the other hand. Therefore the patient and the parents asked to start again the celecoxib medication. During the next weeks, the worsening in behaviour and mood was slowly revised.

As various changes could be made in the above methods and compositions without departing from the scope of the invention, it is intended that all matter contained in the above description shall be interpreted as illustrative and not in a limiting sense.

What is claimed is:

1. A method for the prevention, treatment, or inhibition of a psychiatric disorder in a subject in need of such prevention, treatment, or inhibition,
comprising administering to the subject a COX-2 inhibitor or prodrug thereof.

2. The method according to claim 1 for the treatment of schizophrenia, delusional disorders, affective disorders, autism and tic disorders.

3. The method according to claim 2 for the treatment of chronic schizophrenic psychoses, schizoaffective psychoses, temporary acute psychotic disorders, depressive episodes, recurring depressive episodes, manic episodes and bipolar affective disorders.

4. The method according to claim 1, wherein the amount of the COX-2 inhibitor or prodrug thereof which is administered to the subject comprises a therapeutic amount.

5. The method according to claim 1, wherein the COX-2 inhibitor is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

6. The method according to claim 5, wherein celecoxib or a pharmaceutically acceptable salt thereof is used as COX-2 inhibitor.

7. The method according to claim 6, wherein celecoxib or a pharmaceutically acceptable salt thereof is administered to the subject in an amount of 50-1600 mg per day.

8. The method according to claim 7, wherein celecoxib or a pharmaceutically acceptable salt thereof is administered to the subject in an amount of 200-600 mg per day.

9. The method according to claim 8, wherein celecoxib or a pharmaceutically acceptable salt thereof is administered to the subject in an amount of 400 mg per day.

10. The method according to claim 1, wherein the COX-2 inhibitor is administered to the subject enterally or parenterally in one or more dose per day.

11. The method according to claim 1, wherein the subject is a human.

12. A method for the prevention, treatment, or inhibition of a psychiatric disorder in a subject in need of such prevention, treatment, or inhibition,
comprising administering to the subject a neuroleptic agent or an antidepressant and a COX-2 inhibitor or prodrug thereof.

13. The method according to claim 12, wherein the amount of the neuroleptic agent or the antidepressant and the amount of the COX-2 inhibitor or prodrug thereof which are administered to the subject together comprise a therapeutic amount of the combination.

14. The method according to claim 12 for the treatment of schizophrenia, delusional disorders, affective disorders, autism and tic disorders.

15. The method according to claim 14 for the treatment of chronic schizophrenic psychoses, schizoaffective psychoses, temporary acute psychotic disorders, depressive episodes, recurring depressive episodes, manic episodes and bipolar affective disorders.

16. The method according to claim 12, wherein the COX-2 inhibitor is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

17. The method according to claim 16, wherein celecoxib or a pharmaceutically acceptable salt thereof is used as the COX-2 inhibitor.

18. The method according to claim 17, wherein celecoxib or a pharmaceutically acceptable salt thereof is administered to the subject in an amount of 50-1600 mg per day.

19. The method according to claim 18, wherein celecoxib or a pharmaceutically acceptable salt thereof is administered to the subject in an amount of 200-600 mg per day.

20. The method according to claim 12, wherein the neuroleptic is selected from the group consisting of clozapine, olanzapine, ziprasidone, risperidone, aripiprazole, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

21. The method according to claim 20, wherein risperidone or aripiprazole is used as the neuroleptic.

22. The method according to claim 21, wherein risperidone is administered to the subject in an amount of 2-6 mg per day.

23. The method according to claim 22, wherein risperidone is administered to the subject in an amount of 4-5 mg per day.

24. The method according to claim 12, wherein the antidepressant is selected from the group consisting of amitriptyline, amitriptyline oxide, desipramine, dibenzepin, dosulepin, doxepin, chloroimipramine, imipramine, nortriptyline, mianserin, maprotiline, trimipramine, viloxazine, trazodone, nefazodone, mirtazapine, venlafaxine, reboxetine, tranylcypromine, brofaromine, moclobemide, citalopram, paroxetine, fluoxetine, fluvoxamine, sertraline, Hypericum (St. John's Wort), and mixtures thereof.

25. The method according to claim 12, wherein the COX-2 inhibitor and the neuroleptic agent or antidepressant are administered to the subject enterally or parenterally in one or more dose per day.

26. The method according to claim 12, wherein the subject is a human.

27. The method according to claim 12, wherein the neuroleptic agent or antidepressant and the COX-2 inhibitor are administered to the subject substantially simultaneously.

28. The method according to claim 12, wherein the neuroleptic agent or antidepressant and the COX-2 inhibitor are administered sequentially.

29. A composition for the treatment, prevention, or inhibition of a psychiatric disorder comprising a neuroleptic agent or antidepressant and a COX-2 inhibitor or prodrug thereof.

30. The composition according to claim 29, wherein the composition is useful for treating a subject in need of treatment, prevention, or inhibition of a psychiatric disorder, and wherein a dose of the composition constitutes an amount of a neuroleptic agent or antidepressant and an amount of a COX-2 inhibitor or a pharmaceutically acceptable salt or prodrug thereof which together constitute a therapeutically effective amount.

31. The composition according to claim 29, wherein the neuroleptic is selected from the group consisting of clozapine, olanzapine, ziprasidone, risperidone, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, prom azine, triflupromazine, chlorprothixene, droperidol, actophenazine, prochlorperazine, methotrimeprazine, pipotiazine, ziprasidone, hoperidone, zuclopenthixol, and mixtures thereof.

32. The composition according to claim 31, wherein the COX-2 inhibitor is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

33. The composition according to claim 29, wherein the antidepressant is selected from the group consisting of amitriptyline, amitriptyline oxide, desipramine, dibenzepin, dosulepin, doxepin, chloroimipramine, imipramine, nortriptyline, mianserin, maprotiline, trimipramine, viloxazine, trazodone, nefazodone, mirtazapine, venlafaxine, reboxetine, tranylcypromine, brofaromine, moclobemide, citalopram, paroxetine, fluoxetine, fluvoxamine, sertraline, Hypericum (St. John's Wort), and mixtures thereof.

34. A kit that is suitable for use in the treatment, prevention or inhibition of a psychiatric disorder,
comprising a first dosage form comprising a neuroleptic agent or an antidepressant and
second dosage form comprising a COX-2 inhibitor or prodrug thereof,
n quantities which comprise a therapeutically effective amount of the combination of the compounds for the treatment, prevention, or inhibition of a psychiatric disorder for simultaneous, separate or sequential administration.

35. The kit according to claim 34, wherein the neuroleptic is selected from the group consisting of clozapine, olanzapine, ziprasidone, risperidone, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof, and the COX-2 inhibitor is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

36. The kit according to claim 34, wherein the antidepressant is selected from the group consisting of amitriptyline, amitriptyline oxide, desipramine, dibenzepin, dosulepin, doxepin, chloroimipramine, imipramine, nortriptyline, mianserin, maprotiline, trimipramine, viloxazine, trazodone, nefazodone, mirtazapine, venlafaxine, reboxetine, tranylcypromine, brofaromine, moclobemide, citalopram, paroxetine, fluoxetine, fluvoxamine, sertraline, Hypericum (St. John's Wort), and mixtures thereof, and the COX-2 inhibitor is selected from celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

37. The kit according to claim 35, wherein the neuroleptic is risperidone and the COX-2 inhibitor is celecoxib or a pharmaceutically acceptable salt thereof.
